SEQUENCE LISTING (6 sequences)

SEQ ID NO: 1 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - DNA, 2217 bp, Homo sapiens
ccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggaaaaaaaaaaaaaaaaaa

SEQ ID NO: 3 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 4 - DNA, 5966 bp, Homo sapiens
gtgatgttatctgctggcagcagaaggttcgctccgagcggagctccagaagctcctgacaagagaaagacagattgagatagagatagaaagagaaagagagaaagagacagcagagcgagagcgcaagtgaaagaggcaggggagggggatggagaatattagcctgacggtctagggagtcatccaggaacaaactgaggggctgcccggctgcagacaggaggagacagagaggatctattttagggtggcaagtgcctacctaccctaagcgagcaattccacgttggggagaagccagcagaggttgggaaagggtgggagtccaagggagcccctgcgcaaccccctcaggaataaaactccccagccagggtgtcgcaagggctgccgttgtgatccgcagggggtgaacgcaaccgcgacggctgatcgtctgtggctgggttggcgtttggagcaagagaaggaggagcaggagaaggagggagctggaggctggaagcgtttgcaagcggcggcggcagcaacgtggagtaaccaagcgggtcagcgcgcgcccgccagggtgtaggccacggagcgcagctcccagagcaggatccgcgccgcctcagcagcctctgcggcccctgcggcacccgaccgagtaccgagcgccctgcgaagcgcaccctcctccccgcggtgcgctgggctcgcccccagcgcgcgcacacgcacacacacacacacacacacacacgcacgcacacacgtgtgcgcttctctgctccggagctgctgctgctcctgctctcagcgccgcagtggaaggcaggaccgaaccgctccttctttaaatatataaatttcagcccaggtcagcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcagaataagtctgtaagtttttttttttctttttaattgtaaatggttctttgtcagtttagtaaaccagtgaaatgttgaaatgttttgacatgtactggtcaaacttcagaccttaaaatattgctgtatagctatgctataggttttttcctttgttttggtatatgtaaccatacctatattattaaaatagatggatatagaagccagcataattgaaaacacatctgcagatctcttttgcaaactattaaatcaaaacattaactactttatgtgtaatgtgtaaatttttaccatattttttatattctgtaataatgtcaactatgatttagattgacttaaatttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttcacagaaaagatgggttcgagttcagtggtcttcatcttccaagcatcattactaaccaagtcagacgttaacaaatttttatgttaggaaaaggaggaatgttatagatacatagaaaattgaagtaaaatgttttcattttagcaaggatttagggttctaactaaaactcagaatctttattgagttaagaaaagtttctctaccttggtttaatcaatatttttgtaaaatcctattgttattacaaagaggacacttcataggaaacatctttttctttagtcaggtttttaatattcagggggaaattgaaagatatatattttagtcgatttttcaaaaggggaaaaaagtccaggtcagcataagtcattttgtgtatttcactgaagttataaggtttttataaatgttctttgaaggggaaaaggcacaagccaatttttcctatgatcaaaaaattctttctttcctctgagtgagagttatctatatctgaggctaaagtttaccttgctttaataaataatttgccacatcattgcagaagaggtatcctcatgctggggttaatagaatatgtcagtttatcacttgtcgcttatttagctttaaaataaaaattaataggcaaagcaatggaatatttgcagtttcacctaaagagcagcataaggaggcgggaatccaaagtgaagttgtttgatatggtctacttcttttttggaatttcctgaccattaattaaagaattggatttgcaagtttgaaaactggaaaagcaagagatgggatgccataatagtaaacagcccttgtgttggatgtaacccaatcccagatttgagtgtgtgttgattatttttttgtcttccacttttctattatgtgtaaatcacttttatttctgcagacattttcctctcagataggatgacattttgttttgtattattttgtctttcctcatgaatgcactgataatattttaaatgctctattttaagatctcttgaatctgttttttttttttttaatttgggggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaattatttcgatgatatgtggatgtctttttcctatcagtaccatcatcgagtctagaaaacacctgtgatgcaataagactatctcaagctggaaaagtcataccacctttccgattgccctctgtgctttctcccttaaggacagtcacttcagaagtcatgctttaaagcacaagagtcaggccatatccatcaaggatagaagaaatccctgtgccgtctttttattcccttatttattgctatttggtaattgtttgagatttagtttccatccagcttgactgccgaccagaaaaaatgcagagagatgtttgcaccatgctttggctttctggttctatgttctgccaacgccagggccaaaagaactggtctagacagtatcccctgtagccccataacttggatagttgctgagccagccagatataacaagagccacgtgctttctggggttggttgtttgggatcagctacttgcctgtcagtttcactggtaccactgcaccacaaacaaaaaaacccaccctatttcctccaatttttttggctgctacctacaagaccagactcctcaaacgagttgccaatctcttaataaataggattaataaaaaaagtaattgtgactcaaaaaaaaaaaaaa

SEQ ID NO: 5 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 6 - DNA, 3183 bp, Homo sapiens
gacagaagcaatggccgaggcagaagacaagccgaggtgctggtgaccctgggcgtctgagtggatgattggggctgctgcgctcagaggcctgcctccctgccttccaatgcatataaccccacaccccagccaatgaagacgagaggcagcgtgaacaaagtcatttagaaagcccccgaggaagtgtaaacaaaagagaaagcatgaatggagtgcctgagagacaagtgtgtcctgtactgcccccacctttagctgggccagcaactgcccggccctgcttctccccacctactcactggtgatctttttttttttacttttttttcccttttcttttccattctcttttcttattttctttcaaggcaaggcaaggattttgattttgggacccagccatggtccttctgcttcttctttaaaatacccactttctccccatcgccaagcggcgtttggcaatatcagatatccactctatttatttttacctaaggaaaaactccagctcccttcccactcccagctgccttgccacccctcccagccctctgcttgccctccacctggcctgctgggagtcagagcccagcaaaacctgtttagacacatggacaagaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcattttttttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggttcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

(19) United States
(12) Patent Application Publication
(10) Pub. No.: US 2014/0170158 A1
(43) Pub. Date: Jun. 19, 2014

(54) COMPOSITIONS AND METHODS FOR TREATING OR PREVENTING LUNG DISEASES

(71) Applicant: THE JOHNS HOPKINS UNIVERSITY, Baltimore, MD (US)

(72) Inventors: Enid R. Neptune, Baltimore, MD (US); Robert A. Wise, Baltimore, MD (US)

(73) Assignee: The Johns Hopkins University, Baltimore, MD (US)

(21) Appl. No.: 13/717,343

(22) Filed: Dec. 17, 2012

(51) Int. Cl. A61K 39/395; A61K 31/4178

(52) U.S. Cl. CPC A61K 39/3955; A61K 31/4178
USPC 424/158.1; 514/381; 435/375

(57) ABSTRACT

As described below, the present invention features compositions and methods for treating or preventing lung disease (e.g., chronic obstructive pulmonary disease (COPD), emphysema, and other conditions associated with cigarette smoke exposure) are urgently required.

### STATEMENT OF RIGHTS TO INVENTIONS MADE UNDER FEDERALLY SPONSORED RESEARCH

This work was supported by the following grants from the National Institutes of Health, Grant Nos: R01HL085312, R03HL095406-01, and P50HL084945. The government has certain rights in the invention.

### BACKGROUND OF THE INVENTION

Smoking-related lung diseases, especially chronic obstructive pulmonary disease (COPD) and emphysema, are the third leading cause of death in the United States. Treatment options are limited to either symptom relief and/or the elimination of environmental cofactors, such as cigarette smoking. Importantly, despite growing data on the cellular, molecular, and genetic features of the disorder, no novel treatments that can alter the natural history of the disease are currently available. Thus, methods for treating or preventing lung disease are urgently required.

### SUMMARY OF THE INVENTION

As described below, the present invention features compositions and methods for treating or preventing lung disease including, but not limited to, acquired diseases, such as chronic obstructive pulmonary disease (COPD), bronchopulmonary dysplasia (BPD), emphysema, asthma, aging related lung dysfunction and lung conditions associated with cigarette smoke or other environmental exposures, as well as lung manifestations associated with matrix disorders, such as Ehlers Danlos Syndrome and Cutis Laxa are urgently required.

The invention generally provides methods for treating or preventing lung cell damage associated with cigarette smoke or other environmental exposure, the method involving contacting a cell with an effective amount of an agent that inhibits TGF-β signaling. In one embodiment, the cell is a pulmonary cell, endothelial cell, pulmonary endothelial cell, smooth muscle cell, ciliated and unciliated epithelial cell, and/or alveolar cell. In another embodiment, the cell is contacted for a time sufficient to improve lung architecture or lung function. In another embodiment, the time is at least about 3, 6, 9, 12, 18, 24 months or more. In yet another embodiment, the agent is a small compound (e.g., Losartan, Telmesartan, Irbesartan, Candesartan, Eprosartan, Olmesartan, and Vaisartan) polypeptide (e.g., TGF-β antibodies), polynucleotide, or inhibitory nucleic acid molecule (inhibitory nucleic acids targeting TGF-β1, 2, and/or 3).

In another aspect, the invention provides a method of preventing or reducing cell death associated with cigarette smoke-induced cell injury or other environmental exposure, the method involving contacting a cell at risk of cell death with an agent that inhibits TGF-β signaling, thereby preventing or reducing cell death relative to an untreated control cell. In one embodiment, the cell death is necrotic or apoptotic.

In another aspect, the invention provides a method of treating or preventing chronic obstructive pulmonary disease (COPD), emphysema, and other symptoms associated with lung tissue injury in a subject (e.g., human) at risk thereof, the method involving administering to the subject an effective amount of an agent that inhibits TGF-β signaling. In one embodiment, the disease is not COPD. In another embodiment, the agent is not an angiotensin inhibitor or blocker. In another embodiment, the agent is administered in an amount and for a time sufficient (e.g., at least about 6 months, 1 year or more) to improve lung architecture or lung function by at least about 10%, 25%, 50%, 75% or more.

In another aspect, the invention provides a method of treating or preventing a lung disease is acquired lung disease, lung conditions associated with cigarette smoke or other environmental exposures, and lung manifestations associated with matrix disorders, the method involving administering to the subject an effective amount of an agent that inhibits TGF-β signaling and/or an angiotensin receptor type 1 blocker/inhibitor.

In another aspect, the invention provides a composition formulated for inhalation, the composition containing an effective amount of an agent that inhibits TGF-β is any one or more of TGF-β antibodies, small compounds that modulate TGF-β signaling, inhibitory nucleic acids targeting TGF-β, and Alk1 and/or Alk5 inhibitors, and combinations thereof in an excipient formulated for delivery to the lung.

In another aspect, the invention provides a device for delivering an aerosol to the lung containing a composition of any of the above aspects or any other composition of the invention delineated herein.

In another aspect, the invention provides a composition formulated for inhalation, the composition containing an effective amount of an angiotensin receptor type 1 blockers/inhibitor that is any one or more of Losartan, Telmesartan, Irbesartan, Candesartan, Eprosartan, Olmesartan, and Valsartan in an excipient formulated for delivery to the lung.

In another aspect, the invention provides a device for delivering an aerosol to the lung containing a composition of any of the above aspects or any other composition of the invention delineated herein.

In another aspect, the invention provides a packaged pharmaceutical containing a therapeutically effective amount of an agent that inhibits TGF-β that is any one or more of TGF-β antibodies, small compounds that modulate TGF-β signaling, inhibitory nucleic acids targeting TGF-β, and Alk1 and/or Alk5 inhibitors and instructions for use.

In another aspect, the invention provides a packaged pharmaceutical containing a therapeutically effective amount of an agent that is an angiotensin receptor type 1 blockers or inhibitor that is any one or more of Losartan, Telmesartan, Irbesartan, Candesartan, Eprosartan, Olmesartan, and Valsartan labeled for use in preventing or treating cigarette smoke-induced cell injury.

In another aspect, the invention provides a kit for the amelioration of treating or preventing cigarette smoke-induced cell injury containing an agent that inhibits TGF-β signaling and written instructions for use of the kit.

In another aspect, the invention provides a packaged pharmaceutical containing a therapeutically effective amount of an agent that is an angiotensin receptor type 1 blocker or inhibitor that is any one or more of Losartan, Telmesartan, Irbesartan, Candesartan, Eprosartan, Olmesartan, and Valsartan labeled for use in preventing or treating cigarette smoke-induced cell injury.

In various embodiments of any of the above aspects or any other aspect of the invention delineated herein, the acquired lung disease is chronic obstructive pulmonary disease (COPD), bronchopulmonary dysplasia (BPD), emphysema, asthma, and aging related lung dysfunction. In various embodiments of any of the above aspects, the matrix disorder is Ehlers Danlos Syndrome, Cutis Laxa, and/or fibrosis. In still other embodiments of any of the above aspect, the method prevents or ameliorates alveolar injury, airway epithelial hyperplasia, and lung fibrosis. In various embodiments of any of the above aspects, the agent is a TGF-β antagonist is any one or more of TGF-β antibodies, small compounds that modulate TGF-β signaling, inhibitory nucleic acids targeting TGF-β, and Alk1 and/or Alk5 inhibitors or angiotensin receptor type 1 blockers/inhibitors that is any one or more of Losartan, Telmesartan, Irbesartan, Candesartan, Eprosartan, Olmesartan, and Valsartan, and combinations thereof. In other embodiments of any of the above aspects, the method prevents cell death or cell damage of a pulmonary cell, endothelial cell, pulmonary endothelial cell, smooth muscle cell, ciliated and unciliated epithelial cell, and/or alveolar cell. In various embodiments of any of the above aspects, the agent is administered before, during, or after cigarette smoke-induced cell injury. In various embodiments of any of the above aspects, the agent is administered to subjects having or at risk for developing a lung disease that is any one or more of Ehlers Danlos Syndrome, Cutis Laxa, acquired lung disease, bronchopulmonary dysplasia (BPD), aging related lung dysfunction, chronic obstructive pulmonary disease (COPD), emphysema, asthma, alveolar injury, airway epithelial hyperplasia, or fibrosis. In various embodiments of any of the above aspects the agent is formulated for delivery by inhalation. In one embodiment, the cell is a pulmonary cell, endothelial cell, pulmonary endothelial cell, smooth muscle cell, ciliated and unciliated epithelial cell, and/or alveolar cell. In another embodiment, the cell is contacted for a time sufficient to improve lung architecture or lung function. In another embodiment, the time is at least about 3, 6, 9, 12, 18, 24 months or more. In yet another embodiment, the agent is a small compound (e.g., Losartan, Telmesartan, Irbesartan, Candesartan, Eprosartan, Olmesartan, and Valsartan) polypeptide (e.g., TGF-β antibodies), polynucleotide, or inhibitory nucleic acid molecule (inhibitory nucleic acids targeting TGF-β1, 2, and/or 3).

The invention features the use of TGF-β antagonists and angiotensin receptor type 1 blockers/inhibitors for the treatment and prevention of chronic obstructive pulmonary disease (COPD), emphysema, and other symptoms associated with lung tissue injury, including but not limited to alveolar injury with overt emphysema and airway epithelial hyperplasia with fibrosis. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “aerosol” is meant a solution of fine particles in a form that is amenable to inhalation and delivery to the lung. Thus, the invention, and in particular use of an aerosol nebulizer, allows both topical and systemic aerosol drug delivery via either the nasal or the pulmonary route for agents of the invention that can be formulated or prepared in-situ or immediately before use as solution, suspension or emulsion or any other pharmaceutical application system (e.g., nanoparticles). The nebulizer can be modified with respect to the pore size and dimension of the mixing chamber to direct aerosol delivery to the lungs. Therefore, various droplet and particle sizes can be generated which can deliver aerosolized particles with a size distribution between 0.01 um and 15 um. By adjusting various parameters, particularly particle size, but also optionally particle density, inspiratory flow rate, the inspired volume when the aerosol “bolus” is delivered, and the total volume inhaled, specific locations within the respiratory tract, may be targeted.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control cell” is meant a corresponding reference cell. For example, a cell from a healthy individual or a cell that is untreated.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of lung diseases include chronic obstructive pulmonary disease (COPD), emphysema, alveolar injury and airway epithelial hyperplasia with fibrosis, as well as Ehlers Danlos Syndrome, acquired lung disease, bronchopulmonary dysplasia (BPD), and aging related lung dysfunction.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “other environmental exposure” is meant exposure to a chemical or other agent present in the environment that is associated with acute or chronic lung damage or injury.

By “formulated for inhalation” is meant that the agent is in a form and present in an excipient that is suitable for delivery to the lung. Suitable formulations include, but are not limited to, aerosols and nanoparticles whose size permits or facilitates inhalation and delivery to the lung.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibits” is meant produces a measurable decrease in a parameter. For example, by inhibition is meant a 10%, 20%, 30%, 40%, 50%, 75%, 85%, or 100% reduction.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “lung tissue injury” is meant any acute tissue damage that occurs as a result of insult to the lung. For example, a toxic insult, oxidative stress, infection, inflammation, or mechanical injury.

By “lung cell damage” is meant any cellular pathology that occurs in response to acute injury or a chronic condition. Exemplary lung cell damage includes cell death, cellular stress, cellular hyperplasia or metaplasia.

By “marker” is meant any protein or polynucleotide having an alteration in expression level or activity that is associated with a disease or disorder.

The term “nebulizer” as used herein is meant to refer to any device that disperses an agent as an aerosol. In certain examples, the device generates an aerosol comprising particles that are between about 0.01-15 microns in size. In preferred examples, when an agent of the invention is applied to the device, the resulting aerosol contains the agent and can deliver it into the lungs of a subject by normal breathing.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “TGF-β signaling” is meant any downstream effect of TGF-β ligand binding to a TGF receptor. In one embodiment, TGF-β signaling produces measurable effects on, for example, cell growth, lung cell apoptosis, and lung cell functions. In other embodiments, TGF-β signaling also produces measurable effects on p21 (proapoptotic/antiapoptotic), p38 (proapoptotic), JNK (proapoptotic), and akt (antiapoptotic) pathways. In still other embodiments, increased TGF-β1 signaling effects psmad2, which can be measured, for example, using immunoassays/immunoblots. In still other embodiments, connective tissue growth factor (CTGF) and extracellular matrix proteins, including but not limited to, fibronectin, collagen, and elastin are downstream markers that increases in response to TGF-β signaling.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G show a bar graph, a histological section, a bar graph, an immunohistochemically stained section, a bar graph, an immunohistochemically stained section, and a bar graph, respectively. Chronic CS induces TGF-β expression in murine lungs and human COPD lungs. As shown in FIG. 1A, TGF-β induction profile by ELISA analysis in lung lysates from AKR/J mice exposed to 2 weeks of CS. *P<0.01, CS versus RA or CS plus losartan (Los) versus CS. n=3-5 mice per treatment group. FIG. 1B shows representative histologic sections of lungs from mice exposed to RA or chronic CS subjected to immunohistochemical staining for psmad2. The inset shows localized staining in alveolar epithelial cells of CS-exposed mice. Arrowheads denote the site of enhanced staining in airspace (AS) walls of patients with COPD. Original magnification, ×20. n=4-8 mice per treatment group. FIG. 1C shows quantitative immunohistochemistry of psmad2 staining in RA- and CS-exposed mice depicted in B. FIG. 1C shows representative immunohistochemical staining for total TGF-β1 in lung sections from a patient with COPD and a control smoker. Original magnification, ×40. Scale bar: 100 μm. n=10 each of control and COPD tissue sections. LAP-TGF-β1, latency-associated peptide TGF-β complex. (E) Active TGF-β levels in lung lysates from control nonsmokers (Ctrl−Tob) (n=8), control smokers (Ctrl+Tob) (n=6), and smokers with moderate COPD (COPD+Tob) (n=11). FIG. 1F shows representative immunohistochemical staining for psmad2 in lung sections from a patient with moderate COPD and a control smoker (airspace—2 right panels, airway—left panel). FIG. 1G depicts quantitative immunohistochemical staining of psmad2 in airspace compartment and airway compartment in lung sections from patients with moderate COPD and smoking controls normalized to tissue area. n=6-11 in each group. AW, airway.

FIGS. 2A-2C depict two bar graphs and an immunohistochemically stained section, respectively. Chronic cigarette smoke induces TGF-β expression and signaling in C57Bl/6 lungs. FIG. 2A shows a TGF-β induction profile by ELISA analysis in lung lysates from C57Bl/6 mice exposed to two weeks of CS. *p<0.01, **p<0.05, CS versus RA (room air) or CS+Los versus CS. N=3-5 mice per treatment group. FIG. 2B depicts representative histologic sections of lungs from adult C57Bl/6 mice exposed to room air (RA), right panel, or chronic CS, left panel, subjected to immunohistochemical staining for psmad2. 20× magnification. N=4-8 mice per treatment group. FIG. 2C quantitative immunohistochemistry of psmad2 staining in room air and CS-exposed C57Bl/6 mice depicted in S1B.

FIGS. 3A-3B are a gel and a bar graph, respectively, that show cigarette smoke induced alterations in the expression of TGF-β signaling mediators. FIG. 3A shows a representative immunoblot analysis of CTGF and TGFb1 expression in lung lysates from AKR/J mice exposed to 4 mos CS. FIG. 3B shows densitometric quantitation of designated immunoblots. N=6-8 mice per condition.

FIG. 4A-4B are a bar graph and a gel, respectively, showing that cigarette smoke extract treatment of MLE12 cells induces TGF-β signaling. FIG. 4A is a densitometric analysis of psmad2 expression in MLE12 cells upon exposure to 72 h of cigarette smoke extract, based on expression levels present in the gel of FIG. 4B. SF-serum free media, CSE-cigarette smoke extract.

FIGS. 5A-5F are two bar graphs, a photomicrograph, a bar graph, a photomicrograph, and a bar graph, respectively, showing that losartan and TGF-β-neutralizing antibody inhibit chronic CS-induced TGF-β signaling in the lung and attenuate destructive airspace enlargement. FIG. 5A shows a morphometric analysis of airspace dimension assessed by mean linear intercept (MLI) in mice subjected to 1 month, 2 months, and 4 months of CS exposure. n=10-25 mice per treatment group. *P<0.01. FIG. 5B shows a morphometric analysis of airspace dimension in mice subjected to 2 months of RA with drinking water or 2 months of CS exposure with drinking water, concurrent low-dose losartan (LD, 0.6 g/l), high-dose losartan (HD, 1.2 g/l), control antibody, or TGF-β-neutralizing antibody (TGFNAb) (10 mg/kg/wk). *P<0.01, RA versus CS or CS versus CS plus other treatments. n=6-8 mice per treatment group. FIG. 5C depicts representative H&E photomicrographs of lungs from mice subjected to 2 months of CS exposure with or without losartan treatment compared with RA controls. Original magnification, ×20. Scale bar: 200 μm. FIG. 5D shows an airway alveolar attachment count in mice subjected to the designated treatments. n=6-8 mice per treatment group. BM, basement membrane. FIG. 5E depicts representative photomicrographs of lungs subjected to CS compared with RA controls or CS plus losartan stained for psmad2 (brown), a marker of TGF-β signaling (airspace compartment—top panel, airway compartment—bottom panel). Original magnification, ×40. Scale bar: 50 μm. FIG. 5F is a bar graph illustrating quantitative immunohistochemistry of psmad2 staining of lungs from aforementioned treatment groups. n=6-8 mice per treatment or condition. CS+Los, CS plus losartan.

FIG. 6 is a bar graph that shows the effect of Losartan treatment on airspace dimension. Two months of low or high dose Losartan treatment does not affect airspace dimension. *p<0.05 compared with all other treatment groups. N=6-8 mice per condition.

FIG. 7 is a bar graph that shows the effect of Losartan treatment on body weight. Two months of Losartan treatment does not rescue CS-induced weight loss. *p<0.05 compared RA treatment. N=6-8 mice per condition.

FIGS. 8A-8B are two bar graphs that illustrate the effect of losartan treatment on lung mechanics of CS-exposed mice. FIG. 8A shows the total lung capacity of lungs subjected to designated treatments (top). FIG. 8B shows the static lung elastance of mice subjected to designated treatments (bottom). *P<0.05 for CS compared with RA; **P<0.05 for CS and losartan compared with CS. n=6-8 mice per treatment or condition. Data are represented as mean±SEM.

FIGS. 9A-9C are three sets of H&E images paired with a corresponding bar graph that show airway wall thickening and epithelial hyperplasia in chronic CS-exposed mice. FIG. 9A shows representative H&E images of small airways from mice treated with 2 months of RA, CS, CS plus losartan, or CS plus TGF-β-neutralizing antibody (TGFNAb). Original magnification, ×20. Scale bar: 50 μm. Measurement of airway wall thickness of small airways of similar caliber in mice subjected to designated treatments. Data are expressed as mean±SEM. **P<0.01. n=6-8 mice per treatment. FIG. 9B shows representative lung sections of airways from mice in designated treatment groups stained for proliferation marker Ki67. n=4-6 mice per group. Original magnification, ×20. Scale bar: 100 mm. Quantitative immunohistochemistry of Ki67 staining of airway epithelial cells. FIG. 9C shows representative images of trichrome staining of airways from mice in designated treatment groups. Original magnification, ×20. Scale bar: 100 μm. Quantitation of trichrome staining in designated groups normalized to airway perimeter. n=7-9 mice per group.

FIGS. 10A-10F are nitrotyrosine stained lung sections, three bar graphs, TUNEL stained lung sections with a corresponding bar graph, and C3 stained lung sections with a corresponding bar graph, respectively, that show the effect of losartan on CS-induced injury measures. FIG. 10A shows nitrotyrosine (NiTyr) staining (brown) of lung parenchyma (right) and airways (left) of lungs exposed to CS or CS plus losartan. Original magnification, ×40. Scale bar: 50 μm. n=4-6 mice per group. FIG. 10B shows quantitative immunohistochemistry of nitrotyrosine-stained lungs. Staining was normalized to tissue area. n=4-6 mice per group. FIG. 10C shows quantitative immunohistochemistry of macrophage abundance in lungs using MAG3 staining. Staining was normalized to tissue area. n=4-6 mice per group. FIG. 10D shows quantitative immunohistochemistry of lymphocyte abundance in lungs using CD45R staining. Staining was normalized to tissue area. n=4-6 mice per group. FIG. 10E shows representative photomicrographs of TUNEL-stained lungs. Arrowheads denote staining in airspace epithelial cells in CS-exposed lungs. Original magnification, ×20 (top row); ×40 (bottom row). Scale bar: 50 μm. n=4-6 mice per condition or per treatment. Quantitative immunohistochemistry of TUNEL staining reflecting the apoptotic index. Data are represented as mean±SEM. FIG. 10F shows representative photomicrographs of active caspase-3-stained (C3-stained) lungs. The black arrowhead denotes positive staining in type II alveolar epithelial type II cell. The white arrowhead denotes negative staining in nearby type II epithelial cell. The black arrow shows lack of staining in type I alveolar epithelial cell. Original magnification, ×40. Scale bar: 50 μm. n=4-6 mice per condition or per treatment. Quantitative immunohistochemistry of active caspase-3 staining normalized to tissue area. Data are represented as mean±SEM. *P<0.05, **P<0.01.

FIGS. 11A-11D are a zymography of lung extracts, a bar graph, a Western blot, and Hart's stained alveolar samples, respectively, that show the effects of losartan on matrix metalloprotease activity and expression. FIG. 11A shows zymography of lung extracts from representative mice with designated exposures and treatments. The top band (black arrowhead) denotes MMP9, and the lower band (gray arrowhead) denotes MMP2. The positive (+) control data represents recombinant mouse MMP9. The lanes were run on the same gel but are noncontiguous. n=4-8 mice per treatment. FIG. 11B shows densitometry of MMP9 zymography bands. n=4-8 mice per treatment. FIG. 11C shows a Western blot analysis of MMP12 expression in lung lysates from mice exposed to RA, CS, or CS plus losartan. MMP12 and β-actin bands are shown. n=4-6 mice per condition. FIG. 11D shows elastin localization by Hart's stain with and without tartrazine counterstaining. Arrows in the top and middle rows show linear deposition of elastin in alveolar walls of RA-exposed mice, and arrowheads show dense, discontinuous deposition in walls in CS-exposed mice. The latter is improved with losartan treatment (arrow). Note that pale staining in airspaces reflects residual agarose in lungs. Scale bar: 50 μm. n=4-6 mice per condition.

FIGS. 12A-12B are a bar graph and stained lung sections, respectively, that show angiotensin receptor expression in CS-exposed lungs. FIG. 12A shows Real-time PCR quantitation of AT1 (Agtr1a) expression in CS- and CS plus losartan-treated mice compared with that in RA controls. Receptor expression was normalized to Gapdh. Error bars represent SEM. n=4-6 mice per treatment group. FIG. 12B shows representative lung sections stained for AT1 (black) in adult mice subjected to 2 months of RA, CS, or CS plus losartan. The arrowhead in the inset denotes enhanced staining for AT1 in the airspace wall of CS-exposed mice. Scale bar: 50 μm; 25 μm (inset). n=4-6 mice per treatment or condition. Data are represented as mean±SEM.

FIGS. 13A-13E are a graph, an immunoblot, a bar graph, lung sections, and a bar graph, respectively, that show a transcriptional analysis of protective effect of Losartan in CS-exposed mice. FIG. 13A shows a graphic depiction of proportion of genes dysregulated with CS (blue) and corrected with concurrent Losartan treatment (red) utilizing expression profile analysis of whole lung RNA. Selected genes are identified. Bottom panel shows heatmap of dataset. Red-induced genes. Green-repressed genes. FIG. 13B shows representative immunoblotting of activated and total Akt, JNK and p38 in lung lysates from mice in designated groups. FIG. 13C shows densitometric analysis of pAKT normalized to Akt in lung lysates. N=4-6 mice per group. FIG. 13D shows representative immunohistochemistry of activated Akt (top) and activated JNK (bottom) of murine lungs with designated exposures. N=5-8 mice per treatment and per exposure. FIG. 13E shows quantitative immunohistochemistry of pAKT staining in airspaces of mice normalized to tissue area. Data are represented as means plus SEM. N=4-6 mice per condition or per treatment.

### DETAILED DESCRIPTION OF THE INVENTION

The invention features compositions and methods that are useful for the treatment or prevention of lung diseases, including acquired diseases, such as chronic obstructive pulmonary disease (COPD), bronchopulmonary dysplasia (BPD), emphysema, asthma, aging related lung dysfunction and lung conditions associated with cigarette smoke or other environmental exposures, as well as lung manifestations associated with matrix disorders, such as Ehlers Danlos Syndrome and Cutis Laxa.

The invention is based, at least in part, on the discovery that systemic administration of a TGF-β-specific neutralizing antibody normalized TGF-β signaling and alveolar cell death, conferring improved lung architecture and lung mechanics in CS-exposed mice. Use of losartan, an angiotensin receptor type 1 blocker used widely in the clinic and known to antagonize TGF-β signaling, also improved oxidative stress, inflammation, metalloprotease activation and elastin remodeling. Accordingly, the invention provides compositions and methods for inhibiting TGF-β signaling through angiotensin receptor blockade. Such methods attenuate CS-induced lung injury as indicated herein below in an established murine model and provide for TGF-β-targeted therapies for patients with COPD and other cigarette smoke associate conditions, as well as Ehlers Danlos Syndrome, acquired lung disease, bronchopulmonary dysplasia (BPD), aging related lung dysfunction.

### COPD and TGF Signaling

Chronic obstructive pulmonary disease (COPD) is a prevalent smoking-related disease for which no therapies currently exist. Dysregulated TGF signaling is associated with lung pathology in patients with COPD and in animal models of lung injury induced by chronic exposure to cigarette smoke (CS). To determine whether inhibiting TGF-β signaling would protect against CS-induced lung injury, it was first confirmed that TGF-β signaling was induced in the lungs of mice chronically exposed to CS, as well as in COPD patient samples. Importantly, key pathological features of smoking-associated lung disease in patients, e.g., alveolar injury with overt emphysema and airway epithelial hyperplasia with fibrosis, accompanied CS-induced alveolar cell apoptosis caused by enhanced TGF-β signaling in CS-exposed mice.

The pleiotropic cytokine, TGF-β, has distinct effects on lung maturation, homeostasis, and repair mechanisms. Genetic association studies of patients with emphysema and histologic surveys of lungs from patients with COPD of varying severity have both implicated disturbances in TGF-β signaling as important components of disease pathogenesis (6). Whereas increased TGF-β signaling may explain the increased extracellular matrix observed in the distal airways of patients with severe COPD, reduced signaling with suboptimal matrix deposition might compromise repair in the airspace compartment, leading to histologic emphysema. Despite the fact that TGF-β is known to be dysregulated in COPD/emphysema, TGF-β manipulation has not been explored in models of CS-induced parenchymal lung disease.

### Renin-Angiotensin-Aldosterone (RAA) Cascade

The role of the renin-angiotensin-aldosterone (RAA) cascade in the lung is not well described. Apart from known effects on the microvasculature, reflecting the potent vasoconstrictive effects of angiotensin II, enhanced RAA signaling also induces fibrosis in several tissue beds, including the kidney and the myocardium (7, 8). These latter effects reflect the ability of angiotensin to promote TGF-β expression and signaling. Although structural alveolar apoptosis and airway fibrosis are common features of COPD pathogenesis, angiotensin receptor blockade has not as yet been explored in models of COPD/emphysema. As reported in more detail below, two pharmacologic strategies were used for TGF-β modulation in a murine model of CS-induced emphysema. Increased TGF-β signaling in the lungs of mice exposed to CS and the lung parenchyma of patients with moderate COPD. Systemic TGF-β antagonism using either a pan-specific-neutralizing antibody or losartan, an angiotensin receptor blocker, improved airway and airspace architecture and lung function in chronic CS-exposed mice, commensurate with normalized injury measures. These studies provide compelling preclinical data supporting the utility of TGF-β targeting for CS-induced lung injury.

The present invention is readily distinguishable from findings present in the prior art relating to CS-induced lung injury. In contrast to earlier studies which induced lung injury by exposing cells acutely to certain toxins, the present study more closely resembles the effects of chronic CS on lung tissue. In particular, the present invention provides for the prevention and/or treatment of lung architecture alterations due to immunoresponsive cell infiltration and associated inflammation. The present invention also prevents the further deterioration of lung structure by reducing cytokine levels, as well as by reducing the infiltration of immunoresponsive cells in lung tissue.

Not only does the present invention improve lung architecture, it also reduces cell death associated with oxidative stress and/or apoptosis. Importantly, it reduces airspace enlargement, reduces airway wall thickening due to collagen build-up and an increase in smooth muscle cell number. It also prevents or treats narrowed airways associated with an increase in the thickness of extracellular matrix that results in a restrictive collar that constrains the airways. All of these changes more closely reflect the actual mechanisms of CS-induced cell injury.

### Other Lung Diseases

In other embodiments, the invention provides for the treatment or prevention of Ehlers Danlos Syndrome, acquired lung disease, bronchopulmonary dysplasia (BPD), and aging related lung dysfunction, which are lung diseases associated with airway enlargement and/or increased TGF-β-signaling, with TGF-β-signaling antagonists and/or angiotensin receptor type 1 blockers/inhibitors. For example, Bronchopulmonary Dysplasia (BPD) is a prenatal disorder that is prevalent among premature infants as a consequence of occident stress injury. In many children born prematurely (i.e., less than 25-34 weeks gestation), the developing lung never forms properly. Children and adults who are born prematurely may suffer from airway enlargement and chronic symptoms of emphysema throughout their lives. Babies that are very premature (i.e., less than 25 weeks gestation are at very high risk for BPD). Babies that are born at less than 30 weeks gestation are at high risk for BPD, and the risk remains for babies born between 25-30 weeks gestation. Accordingly, the invention provides compositions and methods featuring agents that inhibit TGF-β signaling to prevent BPD in babies born prematurely (e.g., less than 25-36 weeks gestational age) as well as to treat BPD in babies born prematurely (e.g., less than 25-36 weeks gestational age) that still require oxygen support at 36 weeks gestational age.

### Pharmaceutical Compositions

As reported herein, increased TGF signaling is associated with COPD, emphysema, and other conditions associated with cigarette smoke exposure, as well as Ehlers Danlos Syndrome, acquired lung disease, bronchopulmonary dysplasia (BPD), aging related lung dysfunction. Accordingly, the invention provides for compositions comprising TGF-β antagonists and angiotensin receptor blockers (e.g., Losartan, Telmesartan, Irbesartan. Candesartan. Eprosartan, Olmesartan, and Valsartan) that are useful for the treatment or prevention of lung injury and cigarette smoking-related cellular damage. In particular embodiments, agents that act as TGF-β antagonists or angiotensin receptor blockers are proteins, inhibitory polynucleotide, or small molecules. Accordingly, the invention provides therapeutic agents that decrease TGF-β signaling in a lung cell (e.g., TGF-β antibodies, small compounds that modulate TGF-β signaling, inhibitory nucleic acids targeting TGF-β, as well as agents that modulate downstream signaling pathways (e.g., Alk1 and/or Alk5 inhibitors, TGF-β receptor II inhibitors, SMAD inhibitors, e.g., SMAD2/3 inhibitors).

An agent that is an angiotensin receptor blocker or an agent that decreases TGF-β signaling or biological activity (e.g., a TGF-β antagonist or angiotensin blocker) may be administered within a pharmaceutically-acceptable diluents, carrier, or excipient, in unit dosage form. Conventional pharmaceutical practice may be employed to provide suitable formulations or compositions to administer the compounds to patients suffering from a lung disease that is associated with lung cell injury and cigarette smoking-related cellular damage. Administration may begin before, during or after lung disease or cigarette smoke-related cell damage. In one embodiment, a TGF-β antagonist (e.g., TGF-β antibodies, small compounds that modulate TGF-β signaling, inhibitory nucleic acids targeting TGF-β, as well as agents that modulate downstream signaling pathways, such as Alk1 and/or Alk5 inhibitors) or an angiotensin blocker (e.g., Losartan, Telmesartan, Irbesartan, Candesartan, Eprosartan, Olmesartan, and Valsartan) is administered before, during or after diagnosis of a lung disease (e.g., COPD, emphysema, cigarette smoke-related conditions, as well as Ehlers Danlos Syndrome, acquired lung disease, bronchopulmonary dysplasia (BPD), aging related lung dysfunction).

Any appropriate route of administration may be employed, for example, administration may be by inhalation, or parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols. In particular embodiments, the invention provides

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for TGF-β antagonist or angiotensin blockers include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for lung injury and cigarette smoke related cell injury. The preferred dosage of a TGF antagonist or angiotensin blocker of the invention is likely to depend on such variables as the type and extent of the disorder, the overall health status of the particular patient, the formulation of the compound excipients, and its route of administration.

With respect to a subject having lung disease or cigarette smoke-related cellular damage, an effective amount is sufficient to decrease TGF-β signaling or reduce angiotensin receptor activity, or otherwise protect a lung cell, lung tissue or organism from damage or death. Generally, doses of TGF-β antagonist or angiotensin blockers would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects.

The present invention provides methods of treating lung disease or cigarette smoke-related cellular damage or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a lung disease or cigarette smoke-related cellular damage or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, such as a TGF-β antagonist (e.g., TGF-β antibodies, small compounds that modulate TGF-β signaling, inhibitory nucleic acids targeting TGF-β, as well as agents that modulate downstream signalling pathways, such as Alk1 and/or Alk5 inhibitors) or an angiotensin blocker (e.g., Losartan, Telmesartan, Irbesartan, Candesartan, Eprosartan, Olmesartan, and Valsartan), or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which lung disease or cigarette smoke-related cellular damage may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with lung disease or cigarette smoke-related cellular damage, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Inhibitory Nucleic Acids

Inhibitory nucleic acid molecules are those oligonucleotides that inhibit the expression or activity of a TGF-β polypeptide. Such oligonucleotides include single and double stranded nucleic acid molecules (e.g., DNA, RNA, and analogs thereof) that bind a nucleic acid molecule that encodes a TGF-β polypeptide (e.g., antisense molecules, siRNA, shRNA) as well as nucleic acid molecules that bind directly to a TGF-β polypeptide to modulate its biological activity (e.g., aptamers).

In one embodiment, an inhibitory nucleic acid molecule inhibits the expression or activity of a polynucleotide encoding a TGF-β polypeptide (UniProtKB/Swiss-Prot: P01137; NCBI Ref: NP_{—}000651). The sequence of an exemplary human TGF-β polypeptide follows:

```
MPPSGLRLLL LLLPLLWLLV LTPGRPAAGL STCKTIDMEL VKRKRIEAIR GQILSKLRLA
                70 80 90 100 110 120
SPPSQGEVPP GPLPEAVLAL YNSTRDRVAG ESAEPEPEPE ADYYAKEVTR VLMVETHNEI
              130 140 150 160 170 180
YDKFKQSTHS IYMFFNTSEL REAVPEPVLL SRAELRLLRL KLKVEQHVEL YQKYSNNSWR
              190 200 210 220 230 240
YLSNRLLAPS DSPEWLSFDV TGVVRQWLSR GGEIEGFRLS AHCSCDSRDN TLQVDINGFT
              250 260 270 280 290 300
TGRRGDLATI HGMNRPFLLL MATPLERAQH LQSSRHRRAL DTNYCFSSTE KNCCVRQLYI
              310 320 330 340 350 360
DFRKDLGWKW IHEPKGYHAN FCLGPCPYIW SLDTQYSKVL ALYNQHNPGA SAAPCCVPQA
              370 380 390 LEPLPIVYYV GRKPKVEQLS NMIVRSCKCS
```

In other embodiments, the invention provides polynucleotides encoding such polypeptides The sequence of an exemplary TGFβ polynucleotide (NCBI Ref: NM_{—}000660) follows:

```
      1 ccccgccgcc gccgcccttc gcgccctggg ccatctccct cccacctccc tccgcggagc
    61 agccagacag cgagggcccc ggccgggggc aggggggacg ccccgtccgg ggcacccccc
  121 cggctctgag ccgcccgcgg ggccggcctc ggcccggagc ggaggaagga gtcgccgagg
  181 agcagcctga ggccccagag tctgagacga gccgccgccg cccccgccac tgcggggagg
  241 agggggagga ggagcgggag gagggacgag ctggtcggga gaagaggaaa aaaacttttg
  301 agacttttcc gttgccgctg ggagccggag gcgcggggac ctcttggcgc gacgctgccc
  361 cgcgaggagg caggacttgg ggaccccaga ccgcctccct ttgccgccgg ggacgcttgc
  421 tccctccctg ccccctacac ggcgtccctc aggcgccccc attccggacc agccctcggg
  481 agtcgccgac ccggcctccc gcaaagactt ttccccagac ctcgggcgca ccccctgcac
  541 gccgccttca tccccggcct gtctcctgag cccccgcgca tcctagaccc tttctcctcc
  601 aggagacgga tctctctccg acctgccaca gatcccctat tcaagaccac ccaccttctg
  661 gtaccagatc gcgcccatct aggttatttc cgtgggatac tgagacaccc ccggtccaag
  721 cctcccctcc accactgcgc ccttctccct gaggacctca gctttccctc gaggccctcc
  781 taccttttgc cgggagaccc ccagcccctg caggggcggg gcctccccac cacaccagcc
  841 ctgttcgcgc tctcggcagt gccggggggc gccgcctccc ccatgccgcc ctccgggctg
  901 cggctgctgc cgctgctgct accgctgctg tggctactgg tgctgacgcc tggccggccg
  961 gccgcgggac tatccacctg caagactatc gacatggagc tggtgaagcg gaagcgcatc
1021 gaggccatcc gcggccagat cctgtccaag ctgcggctcg ccagcccccc gagccagggg
1081 gaggtgccgc ccggcccgct gcccgaggcc gtgctcgccc tgtacaacag cacccgcgac
1141 cgggtggccg gggagagtgc agaaccggag cccgagcctg aggccgacta ctacgccaag
1201 gaggtcaccc gcgtgctaat ggtggaaacc cacaacgaaa tctatgacaa gttcaagcag
1261 agtacacaca gcatatatat gttcttcaac acatcagagc tccgagaagc ggtacctgaa
1321 cccgtgttgc tctcccgggc agagctgcgt ctgctgaggc tcaagttaaa agtggagcag
1381 cacgtggagc tgtaccagaa atacagcaac aattcctggc gatacctcag caaccggctg
1441 ctggcaccca gcgactcgcc agagtggtta tcttttgatg tcaccggagt tgtgcggcag
1501 tggttgagcc gtggagggga aattgagggc tttcgcctta gcgcccactg ctcctgtgac
1561 agcagggata acacactgca agtggacatc aacgggttca ctaccggccg ccgaggtgac
1621 ctggccacca ttcatggcat gaaccggcct ttcctgcttc tcatggccac cccgctggag
1681 agggcccagc atctgcaaag ctcccggcac cgccgagccc tggacaccaa ctattgcttc
1741 agctccacgg agaagaactg ctgcgtgcgg cagctgtaca ttgacttccg caaggacctc
1801 ggctggaagt ggatccacga gcccaagggc taccatgcca acttctgcct cgggccctgc
1861 ccctacattt ggagcctgga cacgcagtac agcaaggtcc tggccctgta caaccagcat
1921 aacccgggcg cctcggcggc gccgtgctgc gtgccgcagg cgctggagcc gctgcccatc
1981 gtgtactacg tgggccgcaa gcccaaggtg gagcagctgt ccaacatgat cgtgcgctcc
2041 tgcaagtgca gctgaggtcc cgccccgccc cgccccgccc cggcaggccc ggccccaccc
2101 cgccccgccc ccgctgcctt gcccatgggg gctgtattta aggacacccg tgccccaagc
2161 ccacctgggg ccccattaaa gatggagaga ggactgcgga aaaaaaaaaa aaaaaaa
```

In one embodiment, an inhibitory nucleic acid molecule inhibits the expression or activity of a polynucleotide encoding a TGF-β2 polypeptide (UniProtKB/Swiss-Prot: P61812). The sequence of an exemplary human TGF-β2 polypeptide follows:

```
                10 20 30 40 50 60
MHYCVLSAFL ILHLVTVALS LSTCSTLDMD QFMRKRIEAI RGQILSKLKL TSPPEDYPEP
                70 80 90 100 110 120
EEVPPEVISI YNSTRDLLQE KASRRAAACE RERSDEEYYA KEVYKIDMPP FFPSENAIPP
              130 140 150 160 170 180
TFYRPYFRIV RFDVSAMEKN ASNLVKAEFR VFRLQNPKAR VPEQRIELYQ ILKSKDLTSP
              190 200 210 220 230 240
TQRYIDSKVV KTRAEGEWLS FDVTDAVHEW LHHKDRNLGF KISLHCPCCT FVPSNNYIIP
              250 260 270 280 290 300
NKSEELEARF AGIDGTSTYT SGDQKTIKST RKKNSGKTPH LLLMLLPSYR LESQQTNRRK
              310 320 330 340 350 360
KRALDAAYCF RNVQDNCCLR PLYIDFKRDL GWKWIHEPKG YNANFCAGAC PYLWSSDTQH
              370 380 390 400 410
SRVLSLYNTI NPEASASPCC VSQDLEPLTI LYYIGKTPKI EQLSNMIVKS CKCS
```

In other embodiments, the invention provides polynucleotides encoding such polypeptides. The sequence of an exemplary TGF-β2 polynucleotide (NCBI Ref: NM_{—}001135599.2) follows:

>gi|305682568|ref|NM_001135599.2| Homo sapiens transforming growth
factor, beta 2 (TGFB2), transcript variant 1, mRNA
GTGATGTTATCTGCTGGCAGCAGAAGGTTCGCTCCGAGCGGAGCTCCAGAAGCTCCTGACAAGAGAAAGA
CAGATTGAGATAGAGATAGAAAGAGAAAGAGAGAAAGAGACAGCAGAGCGAGAGCGCAAGTGAAAGAGGC
AGGGGAGGGGGATGGAGAATATTAGCCTGACGGTCTAGGGAGTCATCCAGGAACAAACTGAGGGGCTGCC
CGGCTGCAGACAGGAGGAGACAGAGAGGATCTATTTTAGGGTGGCAAGTGCCTACCTACCCTAAGCGAGC
AATTCCACGTTGGGGAGAAGCCAGCAGAGGTTGGGAAAGGGTGGGAGTCCAAGGGAGCCCCTGCGCAACC
CCCTCAGGAATAAAACTCCCCAGCCAGGGTGTCGCAAGGGCTGCCGTTGTGATCCGCAGGGGGTGAACGC
AACCGCGACGGCTGATCGTCTGTGGCTGGGTTGGCGTTTGGAGCAAGAGAAGGAGGAGCAGGAGAAGGAG
GGAGCTGGAGGCTGGAAGCGTTTGCAAGCGGCGGCGGCAGCAACGTGGAGTAACCAAGCGGGTCAGCGCG
CGCCCGCCAGGGTGTAGGCCACGGAGCGCAGCTCCCAGAGCAGGATCCGCGCCGCCTCAGCAGCCTCTGC
GGCCCCTGCGGCACCCGACCGAGTACCGAGCGCCCTGCGAAGCGCACCCTCCTCCCCGCGGTGCGCTGGG
CTCGCCCCCAGCGCGCGCACACGCACACACACACACACACACACACACGCACGCACACACGTGTGCGCTT
CTCTGCTCCGGAGCTGCTGCTGCTCCTGCTCTCAGCGCCGCAGTGGAAGGCAGGACCGAACCGCTCCTTC
TTTAAATATATAAATTTCAGCCCAGGTCAGCCTCGGCGGCCCCCCTCACCGCGCTCCCGGCGCCCCTCCC
GTCAGTTCGCCAGCTGCCAGCCCCGGGACCTTTTCATCTCTTCCCTTTTGGCCGGAGGAGCCGAGTTCAG
ATCCGCCACTCCGCACCCGAGACTGACACACTGAACTCCACTTCCTCCTCTTAAATTTATTTCTACTTAA
TAGCCACTCGTCTCTTTTTTTCCCCATCTCATTGCTCCAAGAATTTTTTTCTTCTTACTCGCCAAAGTCA
GGGTTCCCTCTGCCCGTCCCGTATTAATATTTCCACTTTTGGAACTACTGGCCTTTTCTTTTTAAAGGAA
TTCAAGCAGGATACGTTTTTCTGTTGGGCATTGACTAGATTGTTTGCAAAAGTTTCGCATCAAAAACAAC
AACAACAAAAAACCAAACAACTCTCCTTGATCTATACTTTGAGAATTGTTGATTTCTTTTTTTTATTCTG
ACTTTTAAAAACAACTTTTTTTTCCACTTTTTTAAAAAATGCACTACTGTGTGCTGAGCGCTTTTCTGAT
CCTGCATCTGGTCACGGTCGCGCTCAGCCTGTCTACCTGCAGCACACTCGATATGGACCAGTTCATGCGC
AAGAGGATCGAGGCGATCCGCGGGCAGATCCTGAGCAAGCTGAAGCTCACCAGTCCCCCAGAAGACTATC
CTGAGCCCGAGGAAGTCCCCCCGGAGGTGATTTCCATCTACAACAGCACCAGGGACTTGCTCCAGGAGAA
GGCGAGCCGGAGGGCGGCCGCCTGCGAGCGCGAGAGGAGCGACGAAGAGTACTACGCCAAGGAGGTTTAC
AAAATAGACATGCCGCCCTTCTTCCCCTCCGAAACTGTCTGCCCAGTTGTTACAACACCCTCTGGCTCAG
TGGGCAGCTTGTGCTCCAGACAGTCCCAGGTGCTCTGTGGGTACCTTGATGCCATCCCGCCCACTTTCTA
CAGACCCTACTTCAGAATTGTTCGATTTGACGTCTCAGCAATGGAGAAGAATGCTTCCAATTTGGTGAAA
GCAGAGTTCAGAGTCTTTCGTTTGCAGAACCCAAAAGCCAGAGTGCCTGAACAACGGATTGAGCTATATC
AGATTCTCAAGTCCAAAGATTTAACATCTCCAACCCAGCGCTACATCGACAGCAAAGTTGTGAAAACAAG
AGCAGAAGGCGAATGGCTCTCCTTCGATGTAACTGATGCTGTTCATGAATGGCTTCACCATAAAGACAGG
AACCTGGGATTTAAAATAAGCTTACACTGTCCCTGCTGCACTTTTGTACCATCTAATAATTACATCATCC
CAAATAAAAGTGAAGAACTAGAAGCAAGATTTGCAGGTATTGATGGCACCTCCACATATACCAGTGGTGA
TCAGAAAACTATAAAGTCCACTAGGAAAAAAAACAGTGGGAAGACCCCACATCTCCTGCTAATGTTATTG
CCCTCCTACAGACTTGAGTCACAACAGACCAACCGGCGGAAGAAGCGTGCTTTGGATGCGGCCTATTGCT
TTAGAAATGTGCAGGATAATTGCTGCCTACGTCCACTTTACATTGATTTCAAGAGGGATCTAGGGTGGAA
ATGGATACACGAACCCAAAGGGTACAATGCCAACTTCTGTGCTGGAGCATGCCCGTATTTATGGAGTTCA
GACACTCAGCACAGCAGGGTCCTGAGCTTATATAATACCATAAATCCAGAAGCATCTGCTTCTCCTTGCT
GCGTGTCCCAAGATTTAGAACCTCTAACCATTCTCTACTACATTGGCAAAACACCCAAGATTGAACAGCT
TTCTAATATGATTGTAAAGTCTTGCAAATGCAGCTAAAATTCTTGGAAAAGTGGCAAGACCAAAATGACA
ATGATGATGATAATGATGATGACGACGACAACGATGATGCTTGTAACAAGAAAACATAAGAGAGCCTTGG
TTCATCAGTGTTAAAAAATTTTTGAAAAGGCGGTACTAGTTCAGACACTTTGGAAGTTTGTGTTCTGTTT
GTTAAAACTGGCATCTGACACAAAAAAAGTTGAAGGCCTTATTCTACATTTCACCTACTTTGTAAGTGAG
AGAGACAAGAAGCAAATTTTTTTTAAAGAAAAAAATAAACACTGGAAGAATTTATTAGTGTTAATTATGT
GAACAACGACAACAACAACAACAACAACAAACAGGAAAATCCCATTAAGTGGAGTTGCTGTACGTACCGT
TCCTATCCCGCGCCTCACTTGATTTTTCTGTATTGCTATGCAATAGGCACCCTTCCCATTCTTACTCTTA
GAGTTAACAGTGAGTTATTTATTGTGTGTTACTATATAATGAACGTTTCATTGCCCTTGGAAAATAAAAC
AGGTGTATAAAGTGGAGACCAAATACTTTGCCAGAAACTCATGGATGGCTTAAGGAACTTGAACTCAAAC
GAGCCAGAAAAAAAGAGGTCATATTAATGGGATGAAAACCCAAGTGAGTTATTATATGACCGAGAAAGTC
TGCATTAAGATAAAGACCCTGAAAACACATGTTATGTATCAGCTGCCTAAGGAAGCTTCTTGTAAGGTCC
AAAAACTAAAAAGACTGTTAATAAAAGAAACTTTCAGTCAGAATAAGTCTGTAAGTTTTTTTTTTTCTTT
TTAATTGTAAATGGTTCTTTGTCAGTTTAGTAAACCAGTGAAATGTTGAAATGTTTTGACATGTACTGGT
CAAACTTCAGACCTTAAAATATTGCTGTATAGCTATGCTATAGGTTTTTTCCTTTGTTTTGGTATATGTA
ACCATACCTATATTATTAAAATAGATGGATATAGAAGCCAGCATAATTGAAAACACATCTGCAGATCTCT
TTTGCAAACTATTAAATCAAAACATTAACTACTTTATGTGTAATGTGTAAATTTTTACCATATTTTTTAT
ATTCTGTAATAATGTCAACTATGATTTAGATTGACTTAAATTTGGGCTCTTTTTAATGATCACTCACAAA
TGTATGTTTCTTTTAGCTGGCCAGTACTTTTGAGTAAAGCCCCTATAGTTTGACTTGCACTACAAATGCA
TTTTTTTTTTAATAACATTTGCCCTACTTGTGCTTTGTGTTTCTTTCATTATTATGACATAAGCTACCTG
GGTCCACTTGTCTTTTCTTTTTTTTGTTTCACAGAAAAGATGGGTTCGAGTTCAGTGGTCTTCATCTTCC
AAGCATCATTACTAACCAAGTCAGACGTTAACAAATTTTTATGTTAGGAAAAGGAGGAATGTTATAGATA
CATAGAAAATTGAAGTAAAATGTTTTCATTTTAGCAAGGATTTAGGGTTCTAACTAAAACTCAGAATCTT
TATTGAGTTAAGAAAAGTTTCTCTACCTTGGTTTAATCAATATTTTTGTAAAATCCTATTGTTATTACAA
AGAGGACACTTCATAGGAAACATCTTTTTCTTTAGTCAGGTTTTTAATATTCAGGGGGAAATTGAAAGAT
ATATATTTTAGTCGATTTTTCAAAAGGGGAAAAAAGTCCAGGTCAGCATAAGTCATTTTGTGTATTTCAC
TGAAGTTATAAGGTTTTTATAAATGTTCTTTGAAGGGGAAAAGGCACAAGCCAATTTTTCCTATGATCAA
AAAATTCTTTCTTTCCTCTGAGTGAGAGTTATCTATATCTGAGGCTAAAGTTTACCTTGCTTTAATAAAT
AATTTGCCACATCATTGCAGAAGAGGTATCCTCATGCTGGGGTTAATAGAATATGTCAGTTTATCACTTG
TCGCTTATTTAGCTTTAAAATAAAAATTAATAGGCAAAGCAATGGAATATTTGCAGTTTCACCTAAAGAG
CAGCATAAGGAGGCGGGAATCCAAAGTGAAGTTGTTTGATATGGTCTACTTCTTTTTTGGAATTTCCTGA
CCATTAATTAAAGAATTGGATTTGCAAGTTTGAAAACTGGAAAAGCAAGAGATGGGATGCCATAATAGTA
AACAGCCCTTGTGTTGGATGTAACCCAATCCCAGATTTGAGTGTGTGTTGATTATTTTTTTGTCTTCCAC
TTTTCTATTATGTGTAAATCACTTTTATTTCTGCAGACATTTTCCTCTCAGATAGGATGACATTTTGTTT
TGTATTATTTTGTCTTTCCTCATGAATGCACTGATAATATTTTAAATGCTCTATTTTAAGATCTCTTGAA
TCTGTTTTTTTTTTTTTTAATTTGGGGGTTCTGTAAGGTCTTTATTTCCCATAAGTAAATATTGCCATGG
GAGGGGGGTGGAGGTGGCAAGGAAGGGGTGAAGTGCTAGTATGCAAGTGGGCAGCAATTATTTTTGTGTT
AATCAGCAGTACAATTTGATCGTTGGCATGGTTAAAAAATGGAATATAAGATTAGCTGTTTTGTATTTTG
ATGACCAATTACGCTGTATTTTAACACGATGTATGTCTGTTTTTGTGGTGCTCTAGTGGTAAATAAATTA
TTTCGATGATATGTGGATGTCTTTTTCCTATCAGTACCATCATCGAGTCTAGAAAACACCTGTGATGCAA
TAAGACTATCTCAAGCTGGAAAAGTCATACCACCTTTCCGATTGCCCTCTGTGCTTTCTCCCTTAAGGAC
AGTCACTTCAGAAGTCATGCTTTAAAGCACAAGAGTCAGGCCATATCCATCAAGGATAGAAGAAATCCCT
GTGCCGTCTTTTTATTCCCTTATTTATTGCTATTTGGTAATTGTTTGAGATTTAGTTTCCATCCAGCTTG
ACTGCCGACCAGAAAAAATGCAGAGAGATGTTTGCACCATGCTTTGGCTTTCTGGTTCTATGTTCTGCCA
ACGCCAGGGCCAAAAGAACTGGTCTAGACAGTATCCCCTGTAGCCCCATAACTTGGATAGTTGCTGAGCC
AGCCAGATATAACAAGAGCCACGTGCTTTCTGGGGTTGGTTGTTTGGGATCAGCTACTTGCCTGTCAGTT
TCACTGGTACCACTGCACCACAAACAAAAAAACCCACCCTATTTCCTCCAATTTTTTTGGCTGCTACCTA
CAAGACCAGACTCCTCAAACGAGTTGCCAATCTCTTAATAAATAGGATTAATAAAAAAAGTAATTGTGAC
TCAAAAAAAAAAAAAA

In one embodiment, an inhibitory nucleic acid molecule inhibits the expression or activity of a polynucleotide encoding a TGF-β3 polypeptide (UniProtKB/Swiss-Prot: P10600). The sequence of an exemplary human TGF-β3 polypeptide follows:

```
                10 20 30 40 50 60
MKMHLQRALV VLALLNFATV SLSLSTCTTL DFGHIKKKRV EAIRGQILSK LRLTSPPEPT
                70 80 90 100 110 120
VMTHVPYQVL ALYNSTRELL EEMHGEREEG CTQENTESEY YAKEIHKFDM IQGLAEHNEL
              130 140 150 160 170 180
AVCPKGITSK VFRFNVSSVE KNRTNLFRAE FRVLRVPNPS SKRNEQRIEL FQILRPDEHI
              190 200 210 220 230 240
AKQRYIGGKN LPTRGTAEWL SFDVTDTVRE WLLRRESNLG LEISIHCPCH TFQPNGDILE
              250 260 270 280 290 300
NIHEVMEIKF KGVDNEDDHG RGDLGRLKKQ KDHHNPHLIL MMIPPHRLDN PGQGGQRKKR
              310 320 330 340 350 360
ALDTNYCFRN LEENCCVRPL YIDFRQDLGW KWVHEPKGYY ANFCSGPCPY LRSADTTHST
              370 380 390 400 410
VLGLYNTLNP EASASPCCVP QDLEPLTILY YVGRTPKVEQ LSNMVVKSCK CS
```

In other embodiments, the invention provides polynucleotides encoding such polypeptides. The sequence of an exemplary TGF-β3 polynucleotide (NCBI Ref: NM_{—}003239.2) follows:

>gi|169790812|ref|NM_003239.2| Homo sapiens transforming growth
factor, beta 3 (TGFB3), mRNA
GACAGAAGCAATGGCCGAGGCAGAAGACAAGCCGAGGTGCTGGTGACCCTGGGCGTCTGAGTGGATGATT
GGGGCTGCTGCGCTCAGAGGCCTGCCTCCCTGCCTTCCAATGCATATAACCCCACACCCCAGCCAATGAA
GACGAGAGGCAGCGTGAACAAAGTCATTTAGAAAGCCCCCGAGGAAGTGTAAACAAAAGAGAAAGCATGA
ATGGAGTGCCTGAGAGACAAGTGTGTCCTGTACTGCCCCCACCTTTAGCTGGGCCAGCAACTGCCCGGCC
CTGCTTCTCCCCACCTACTCACTGGTGATCTTTTTTTTTTTACTTTTTTTTCCCTTTTCTTTTCCATTCT
CTTTTCTTATTTTCTTTCAAGGCAAGGCAAGGATTTTGATTTTGGGACCCAGCCATGGTCCTTCTGCTTC
TTCTTTAAAATACCCACTTTCTCCCCATCGCCAAGCGGCGTTTGGCAATATCAGATATCCACTCTATTTA
TTTTTACCTAAGGAAAAACTCCAGCTCCCTTCCCACTCCCAGCTGCCTTGCCACCCCTCCCAGCCCTCTG
CTTGCCCTCCACCTGGCCTGCTGGGAGTCAGAGCCCAGCAAAACCTGTTTAGACACATGGACAAGAATCC
CAGCGCTACAAGGCACACAGTCCGCTTCTTCGTCCTCAGGGTTGCCAGCGCTTCCTGGAAGTCCTGAAGC
TCTCGCAGTGCAGTGAGTTCATGCACCTTCTTGCCAAGCCTCAGTCTTTGGGATCTGGGGAGGCCGCCTG
GTTTTCCTCCCTCCTTCTGCACGTCTGCTGGGGTCTCTTCCTCTCCAGGCCTTGCCGTCCCCCTGGCCTC
TCTTCCCAGCTCACACATGAAGATGCACTTGCAAAGGGCTCTGGTGGTCCTGGCCCTGCTGAACTTTGCC
ACGGTCAGCCTCTCTCTGTCCACTTGCACCACCTTGGACTTCGGCCACATCAAGAAGAAGAGGGTGGAAG
CCATTAGGGGACAGATCTTGAGCAAGCTCAGGCTCACCAGCCCCCCTGAGCCAACGGTGATGACCCACGT
CCCCTATCAGGTCCTGGCCCTTTACAACAGCACCCGGGAGCTGCTGGAGGAGATGCATGGGGAGAGGGAG
GAAGGCTGCACCCAGGAAAACACCGAGTCGGAATACTATGCCAAAGAAATCCATAAATTCGACATGATCC
AGGGGCTGGCGGAGCACAACGAACTGGCTGTCTGCCCTAAAGGAATTACCTCCAAGGTTTTCCGCTTCAA
TGTGTCCTCAGTGGAGAAAAATAGAACCAACCTATTCCGAGCAGAATTCCGGGTCTTGCGGGTGCCCAAC
CCCAGCTCTAAGCGGAATGAGCAGAGGATCGAGCTCTTCCAGATCCTTCGGCCAGATGAGCACATTGCCA
AACAGCGCTATATCGGTGGCAAGAATCTGCCCACACGGGGCACTGCCGAGTGGCTGTCCTTTGATGTCAC
TGACACTGTGCGTGAGTGGCTGTTGAGAAGAGAGTCCAACTTAGGTCTAGAAATCAGCATTCACTGTCCA
TGTCACACCTTTCAGCCCAATGGAGATATCCTGGAAAACATTCACGAGGTGATGGAAATCAAATTCAAAG
GCGTGGACAATGAGGATGACCATGGCCGTGGAGATCTGGGGCGCCTCAAGAAGCAGAAGGATCACCACAA
CCCTCATCTAATCCTCATGATGATTCCCCCACACCGGCTCGACAACCCGGGCCAGGGGGGTCAGAGGAAG
AAGCGGGCTTTGGACACCAATTACTGCTTCCGCAACTTGGAGGAGAACTGCTGTGTGCGCCCCCTCTACA
TTGACTTCCGACAGGATCTGGGCTGGAAGTGGGTCCATGAACCTAAGGGCTACTATGCCAACTTCTGCTC
AGGCCCTTGCCCATACCTCCGCAGTGCAGACACAACCCACAGCACGGTGCTGGGACTGTACAACACTCTG
AACCCTGAAGCATCTGCCTCGCCTTGCTGCGTGCCCCAGGACCTGGAGCCCCTGACCATCCTGTACTATG
TTGGGAGGACCCCCAAAGTGGAGCAGCTCTCCAACATGGTGGTGAAGTCTTGTAAATGTAGCTGAGACCC
CACGTGCGACAGAGAGAGGGGAGAGAGAACCACCACTGCCTGACTGCCCGCTCCTCGGGAAACACACAAG
CAACAAACCTCACTGAGAGGCCTGGAGCCCACAACCTTCGGCTCCGGGCAAATGGCTGAGATGGAGGTTT
CCTTTTGGAACATTTCTTTCTTGCTGGCTCTGAGAATCACGGTGGTAAAGAAAGTGTGGGTTTGGTTAGA
GGAAGGCTGAACTCTTCAGAACACACAGACTTTCTGTGACGCAGACAGAGGGGATGGGGATAGAGGAAAG
GGATGGTAAGTTGAGATGTTGTGTGGCAATGGGATTTGGGCTACCCTAAAGGGAGAAGGAAGGGCAGAGA
ATGGCTGGGTCAGGGCCAGACTGGAAGACACTTCAGATCTGAGGTTGGATTTGCTCATTGCTGTACCACA
TCTGCTCTAGGGAATCTGGATTATGTTATACAAGGCAAGCATTTTTTTTTTTTTTTTAAAGACAGGTTAC
GAAGACAAAGTCCCAGAATTGTATCTCATACTGTCTGGGATTAAGGGCAAATCTATTACTTTTGCAAACT
GTCCTCTACATCAATTAACATCGTGGGTCACTACAGGGAGAAAATCCAGGTCATGCAGTTCCTGGCCCAT
CAACTGTATTGGGCCTTTTGGATATGCTGAACGCAGAAGAAAGGGTGGAAATCAACCCTCTCCTGTCTGC
CCTCTGGGTCCCTCCTCTCACCTCTCCCTCGATCATATTTCCCCTTGGACACTTGGTTAGACGCCTTCCA
GGTCAGGATGCACATTTCTGGATTGTGGTTCCATGCAGCCTTGGGGCATTATGGGTTCTTCCCCCACTTC
CCCTCCAAGACCCTGTGTTCATTTGGTGTTCCTGGAAGCAGGTGCTACAACATGTGAGGCATTCGGGGAA
GCTGCACATGTGCCACACAGTGACTTGGCCCCAGACGCATAGACTGAGGTATAAAGACAAGTATGAATAT
TACTCTCAAAATCTTTGTATAAATAAATATTTTTGGGGCATCCTGGATGATTTCATCTTCTGGAATATTG
TTTCTAGAACAGTAAAAGCCTTATTCTAAGGTG

Ribozymes

Catalytic RNA molecules or ribozymes that include an antisense TGF-β sequence of the present invention can be used to inhibit expression of a TGF-β nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an sirNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of the TGFβ gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of TGF-β expression. In one embodiment, TGF-β expression is reduced in an endothelial cell or an astrocyte. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

In one embodiment, the invention provides methods of treating lung disease (e.g., COPD, emphysema, cigarette smoke-related conditions, as well as Ehlers Danlos Syndrome, acquired lung disease, bronchopulmonary dysplasia (BPD), aging related lung dysfunction) featuring a polynucleotide encoding an inhibitory nucleic acid molecule that targets TGF-β is another therapeutic approach for treating lung disease. Contact with a lung cell or expression of such inhibitory nucleic acid molecules in a lung cell is expected to be useful for ameliorating lung diseases. Such nucleic acid molecules can be delivered to cells of a subject having lung disease. The nucleic acid molecules must be delivered to the cells of a subject in a form in which they can be taken up so that therapeutically effective levels of a inhibitory nucleic acid molecule or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a TGF-β inhibitory nucleic acid molecule, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Ban Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic to a cell of a patient requiring therapy for a lung disease (e.g., COPD, emphysema, cigarette smoke-related conditions, as well as Ehlers Danlos Syndrome, acquired lung disease, bronchopulmonary dysplasia (BPD), aging related lung dysfunction). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

The expression of an inhibitory nucleic acid molecule in a cell can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The dosage of the administered inhibitory nucleic acid molecule depends on a number of factors, including the size and health of the individual patient. For any particular subject, the specific dosage regimes should be adjusted over time according to the individual need and the professional judgment of the person administering or supervising the administration of the compositions.

Other agents useful in the invention are agents that selectively inhibit TGF-β signaling, such as antibodies that selectively bind TGF-β or a TGF-β receptor.

Antibodies that Inhibit TGF-β Signalling

Antibodies useful in the invention include any antibody capable of selectively inhibiting

TGF-β signaling by binding TGF-β or a TGF-β receptor. A polypeptide that “selectively binds” TGF-β or a TGF-β receptor is one that binds TGF-β or a TGF-β receptor, but that does not substantially bind other molecules in a sample, for example, a biological sample. Preferably, such an antibody binds with an affinity constant less than or equal to 10 mM. In various embodiments, the TGF-β or a TGF-β receptor binds its target with an affinity constant that is less than or equal to 1 mM, 100 nM, 10 nM, 1 nM, 0.1 nM, or even less than 0.01 or 0.001 nM. TGF-β or a TGF-β receptor antibodies include polypeptides that when endogenously expressed bind a naturally occurring TGF-β or a TGF-β receptor and fragments thereof.

Antibodies that selectively inhibit TGF-β signaling by binding TGF-β or a TGF-β receptor are useful in the methods of the invention. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cells of interest. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that selectively inhibits TGFβ signaling by binding TGFβ or a TGFβ receptor is monoclonal. Alternatively, the antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing TGF-β or a TGF-β receptor, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a TGF-β or a TGF-β receptor or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding TGF-β or a TGF-β receptor, or immunogenic fragments thereof, can be expressed in cells in vitro, followed by isolation of the receptor and administration of the receptor to a suitable host in which antibodies are raised.

Alternatively, antibodies against TGF-β or a TGF-β receptor may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Pharmaceutical Therapeutics

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the lung disease. Generally, amounts will be in the range of those used for other agents used in the treatment of other diseases associated with lung disease, although in certain instances lower amounts will be needed because of the increased specificity of the compound.

### Formulation of Pharmaceutical Compositions

The administration of a compound for the treatment of lung disease may be by any suitable means that results in a concentration of the therapeutic that, combined with other components, is effective in ameliorating, reducing, or stabilizing lung disease/function. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a lung disease by using carriers or chemical derivatives to deliver the therapeutic agent to a particular cell type (e.g., alveolar cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (inhalation, subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates lung disease, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active anti-lung disease therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutaminine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-lung disease therapeutic substance). The coating may be applied on the solid dosage form in a similar manner as that described in Encyclopedia of Pharmaceutical Technology, supra.

At least two anti-lung disease therapeutics may be mixed together in the tablet, or may be partitioned. In one example, the first active anti-lung disease therapeutic is contained on the inside of the tablet, and the second active anti-lung disease therapeutic is on the outside, such that a substantial portion of the second anti-lung disease therapeutic is released prior to the release of the first anti-lung disease therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-TGF-β therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

The invention provides kits for preventing or treating lung disease or cigarette smoke related cellular damage (e.g., lung fibrosis). In one embodiment, the kit comprises a sterile container that contains a TGF antagonist or angiotensin blocker; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the TGF antagonist or angiotensin blocker in treating or preventing lung disease or cigarette smoke-related cellular damage. Preferably, the kit further comprises any one or more of the reagents described in the assays described herein. In other embodiments, the instructions include at least one of the following: description of the TGF antagonist or angiotensin blocker; methods for using the enclosed materials for the treatment or prevention of a lung disease or cigarette smoke-related cellular damage; precautions; warnings; indications; clinical or research studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### TGF-β Activity is Increased in the Lungs of Mice and Lung Epithelial Cells Exposed to Cigarette Smoke (CS) and in Lungs of Patients with Chronic Obstructive Pulmonary Disease (COPD)

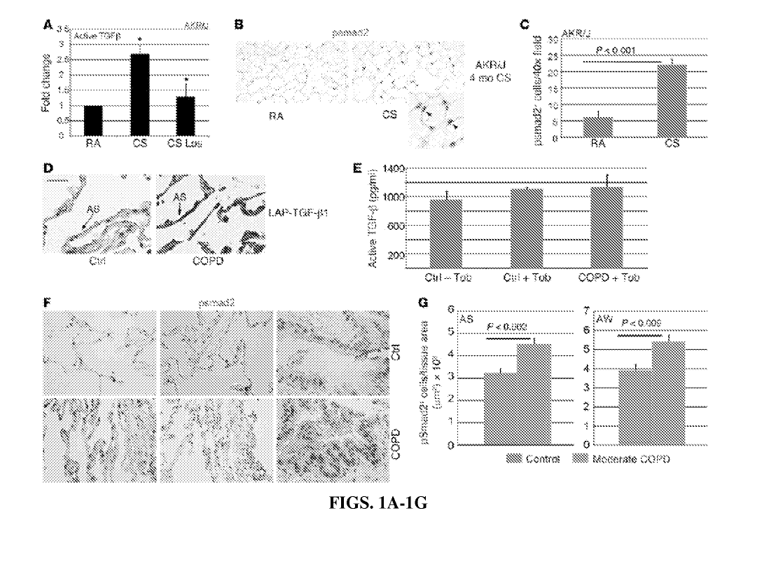

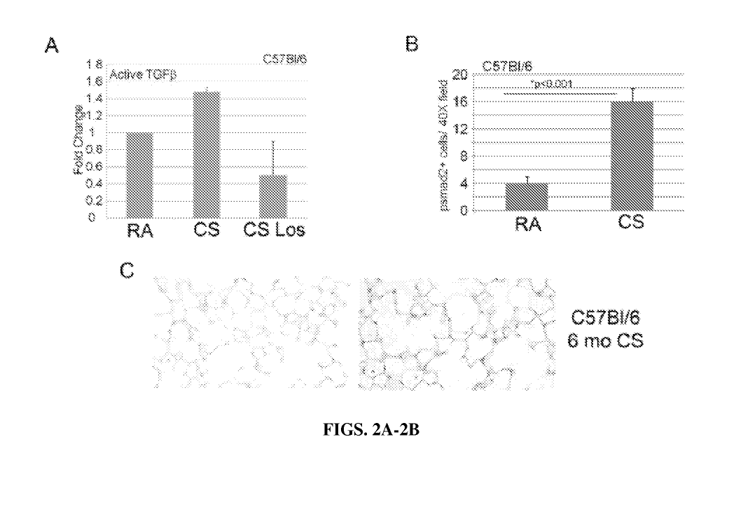

To determine whether CS exposure resulted in elevated levels of active TGF-β, the lungs of 2 strains of mice known to be sensitive to CS were evaluated, and treatment with the angiotensin receptor blocker losartan was assessed to determine whether it normalized this induction of TGF-β. As shown in FIGS. 1 and 2, two weeks of CS exposure significantly induced active TGF-β as shown by ELISA analysis in both AKR/J mice (2.5 fold) and C57BL/6 mice (1.4 fold) (see, e.g., FIGS. 1A and 2A). Concurrent losartan treatment normalized TGF-β in both strains.

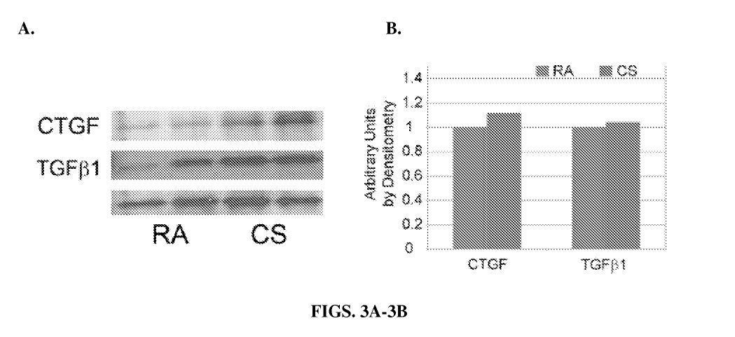

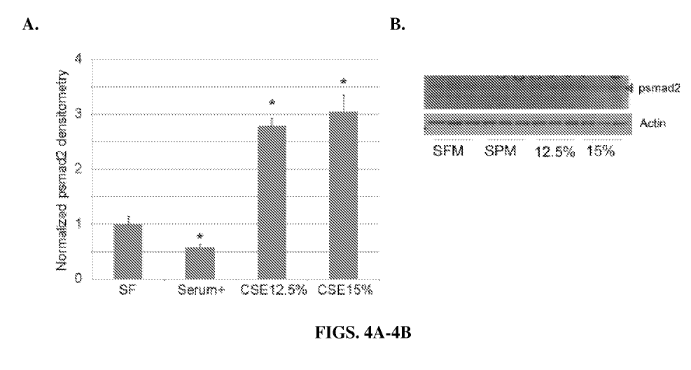

To extend these findings to a chronic CS-induced emphysema model, phosphorylated Smad2 (psmad2) staining, an index of active TGF-β signaling, was evaluated in lung sections from mice that develop emphysema after 4 months of CS exposure, AKR/J mice, and mice that develop emphysema after 6 months of CS exposure, C57BL/6 mice. Psmad2 staining was increased in the lungs of both strains of CS-exposed mice (FIGS. 1B, 1C, 2B, and 2C), primarily in alveolar epithelial cells (See inset, FIG. 1B). Modest elevations of connective tissue growth factor (CTGF), a downstream marker of TGF-β signaling, and TGF-β1 were observed in the lung lysates from AKR/J mice exposed to 4 months of CS (see, e.g., FIGS. 3A-3B). Treatment of murine lung epithelial cells, MLE12 cells, with CS extract (CSE) also induced enhanced TGF-β activation, evident in psmad2 expression by immunoblotting (FIG. 4).

Finally, to extend this observation to clinical COPD, lung samples from at-risk controls (smokers with normal lung function) and patients with moderate COPD were examined. ELISA analysis of active TGF-β1 in lung lysates showed a modest smoking-induced increase in the whole lung levels that was unaffected by COPD status (see, e.g., FIG. 1E). However, increased TGF-β1 and psmad2 were consistently observed in the airspaces of patients with moderate COPD, when compared with those of smoking controls (see, e.g., FIGS. 1D, 1F, and 1G). Patients with moderate COPD were chosen rather than patients with severe COPD in order to avoid the end-stage effects often seen with severe COPD that are punctuated by extensive airspace destruction and overall reduced protein expression. The TGF-β1 in the lungs of these patients with COPD was localized to the alveolar septal walls (similar to that in the murine models) and to inflammatory cells. These data implicate elevated TGF-β signaling as a component of CS-induced lung injury.

### Example 2

### TGF-β Antagonism Improves Airspace Enlargement in Chronic CS-Exposed Mice

The losartan effect on TGF-β signaling after short-term CS exposure suggested that angiotensin receptor blockade might have salutary effects on long-term sequelae of CS exposure. The AKR/J strain was used in subsequent experiments for 2 reasons: (a) to incorporate shorter-term chronic exposures that still generated a measurable airspace lesion and (b) to use an inbred strain that has a CS-induced inflammatory profile more consistent with that of a typical patient with COPD than that of the conventional C57BL/6 model (11). This is a significant advantage over the conventional art, in which most investigators still use the C57BL/6 model that has the potential shortcomings of showing mild lesions with no evidence of airway pathology when exposed to CS.

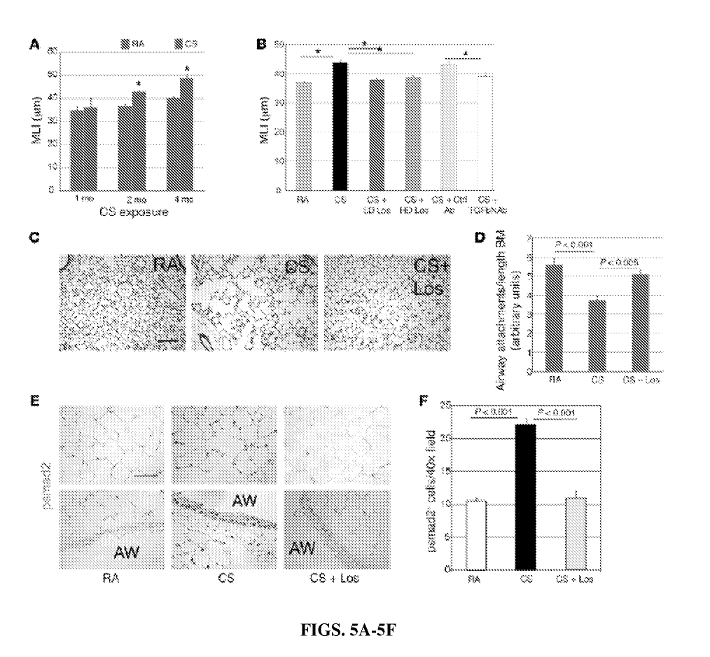

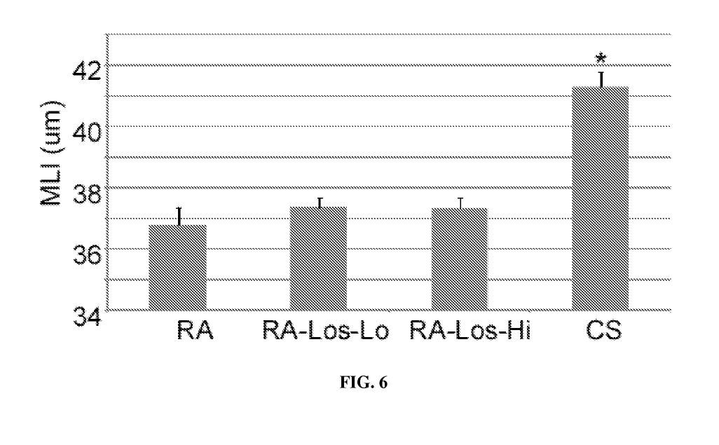

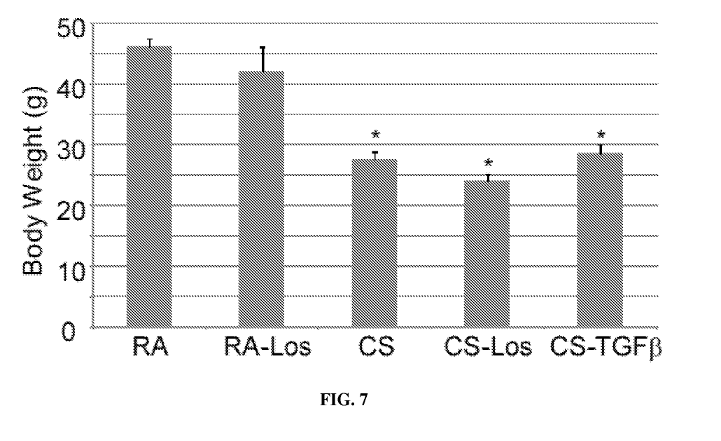

To establish the earliest time point at which an increase in airspace dimension—the signature feature of emphysema—could be observed, AKR/J mice were exposed to CS for 1, 2, and 4 months, and then subjected to morphometric analysis. Although no increase in airspace dimension was observed after of 1 month of exposure, significant emphysema developed after 2 months (see, e.g., FIG. 5A). It should be noted that age-related increases in airspace dimension in room air-exposed (RA-exposed) mice was also observed, a finding recently dissected in another inbred strain but that notably occurs earlier in the AKR/J mice (12). Mice were treated with losartan at 2 doses, 0.6 g/l losartan (low dose) or 1.2 g/l losartan (high dose) in drinking water, concurrent with the CS exposure. A marked reduction in the airspace dimension after 2 months was observed, as shown in FIGS. 5B and 5C. RA-exposed mice treated with the 2 doses of losartan showed no change in airspace caliber or histology compared with those of untreated controls (see, e.g., FIGS. 5B and 6). Assessment of airway attachments, a measure of airspace destruction, showed a significant reduction with CS but recovery with losartan treatment (FIG. 5D). By contrast, CS-induced weight loss was not improved with either losartan or TGF-β-neutralizing antibody treatment (see, e.g., FIG. 7). Losartan treatment of RA-exposed mice did not alter body weight.

To test the hypothesis that these effects were mediated by inhibition of TGF-β, CS-exposed mice were treated with a neutralizing antibody to TGF-β(2, 3). Similar to losartan, TGF-β antagonism with neutralizing antibody given concurrently with CS improved airspace dimension compared with that of CS-exposed mice treated with isotype-matched control antibody (see, e.g., FIG. 5B). RA-exposed mice treated with the neutralizing antibody showed no change in airspace caliber or histology compared with those of untreated controls (data not shown). Phosphorylated smad2 was increased in the alveolar and airway epithelium in CS-exposed mice and normalized with losartan treatment (see, e.g., FIGS. 2E and 2F). Thus, two different strategies targeting TGF-β signaling resulted in improved airspace dimension.

### Example 3

### Losartan Treatment Results in Improved Lung Mechanics and Airway Histology in Chronic CS-Exposed Mice

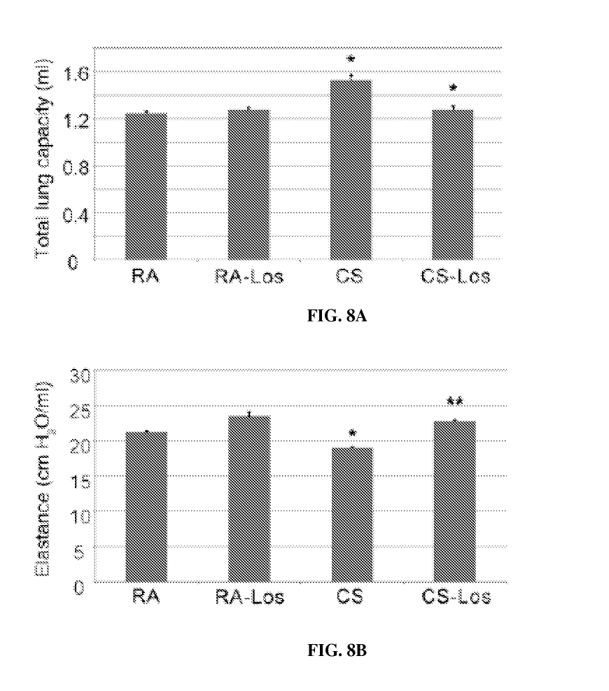

The critical disturbance that drives clinical disease in COPD is the attendant alteration in lung function that follows from altered lung histology. Compared with those of RA-exposed mice, CS-exposed mice had increased lung size and reduced lung elastance, typical physiologic disturbances in emphysema (see, e.g., FIGS. 8A and 8B). Losartan normalized lung size and lung elastance, suggesting that the protective effects apparent by lung histology translated into improved lung function. Notably, losartan treatment of RA-exposed mice did not significantly alter lung mechanics, although there was a trend toward increased elastance.

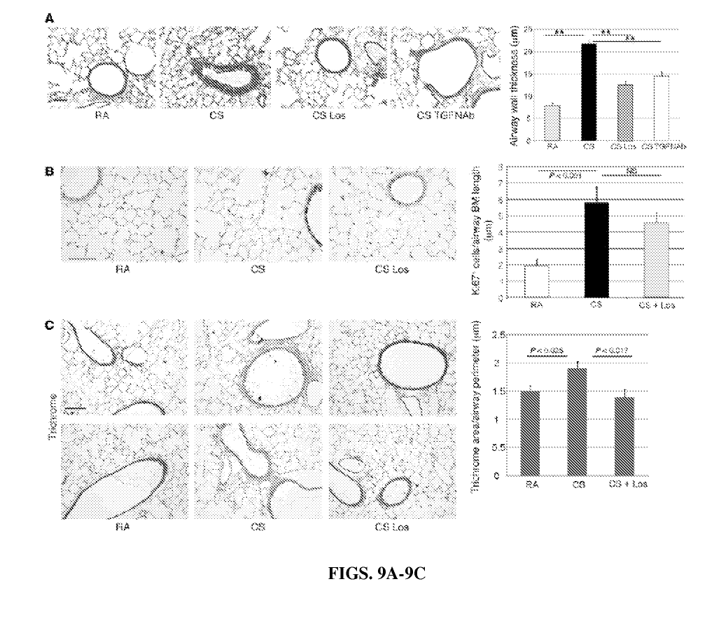

Mice exposed to CS developed mucosal thickening that approximated the epithelial hyperplasia observed in patients with COPD/emphysema (see, e.g., FIG. 9A and ref. 13). Epithelial thickness was measured in airways of similar size in mice exposed to RA, CS, CS plus losartan, and CS plus TGF-β-neutralizing antibody. CS produced a greater than 2-fold increase in airway mucosal thickness (see, e.g., FIG. 9A). Airway epithelial thickening normalized with losartan treatment and TGF-β-neutralizing antibody treatment. No increase in PAS staining (goblet cells) was observed in the CS-exposed airways (data not shown). Ki67 staining of the airway compartment was performed to determine whether the airway thickening represented a proliferative process possibly triggered by CS exposure. An increase in airway epithelial proliferation was observed with CS exposure, with a trend toward reduction with losartan treatment (see, e.g., FIG. 9B). Since TGF-β can induce small airway remodeling, collagen deposition in CS-exposed lungs was examined. While only a minimal increase in collagen deposition was seen in mice exposed to 2 months of CS, a marked increase in peribronchiolar collagen deposition was observed in mice exposed to 3 months of CS (see, e.g., FIG. 9C). Losartan normalized collagen deposition in such mice. The density and abundance of αSMA-producing smooth muscle cells surrounding the small airways was not changed with CS or losartan treatment (data not shown). Without being bound to any particular theory, this airway lesion is believed to be a direct toxic effect of CS that involves TGF-β dysregulation. In summary, airspace enlargement, airway epithelial thickening, peribronchiolar fibrosis, and altered lung mechanics were all ameliorated by losartan treatment and TGF-β antagonism.

### Example 4

### TGF-β Antagonism Improves CS-Induced Oxidative Stress, Inflammation, and Cell Death

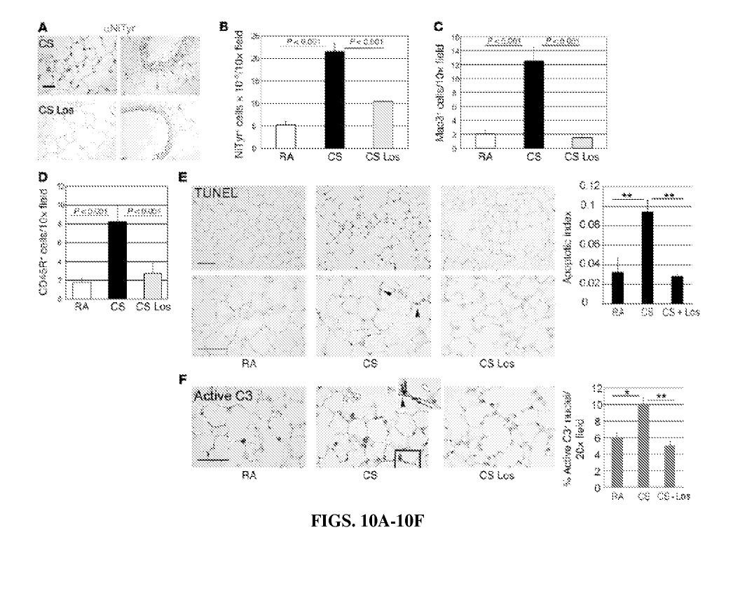

Oxidative stress and inflammation mediate CS-induced lung injury in patients with COPD and murine models of acquired emphysema (14, 15). In AKR/J mice exposed to 2 weeks or 2 months of CS, nitrotyrosine and 8-deoxyguanine immunostaining were increased (see, e.g., FIGS. 10A and 10B, and data not shown), as were alveolar macrophage and lymphocyte numbers (see, e.g., FIGS. 10C and 10D). Of note, we saw no increase in neutrophils in the CS-exposed lungs (data not shown). Losartan treatment normalized oxidative stress and reduced inflammatory cell infiltration into the CS-exposed lungs (see, e.g., FIGS. 10 A-D). TGF-β is known to not only inhibit cellular proliferation, a property observed in various epithelial model systems, but also induce cell death, notably in the alveolar lung cells, as seen in fibrillin-1-deficient mice (3). Reduced airspace epithelial cell proliferation was observed with CS exposure that did not normalize with losartan treatment. By contrast, the enhanced TUNEL and active caspase-3 labeling in the airspace, indicating alveolar epithelial apoptosis, with smoke exposure was attenuated by losartan treatment (see, e.g., FIGS. 10E and 10F).

### Example 5

### Anti-TGF-β Pharmacologics Ameliorate Metalloprotease Activation and Apoptotic Cell Death, which are Key Mechanisms Underlying CS-Induced Airspace Enlargement

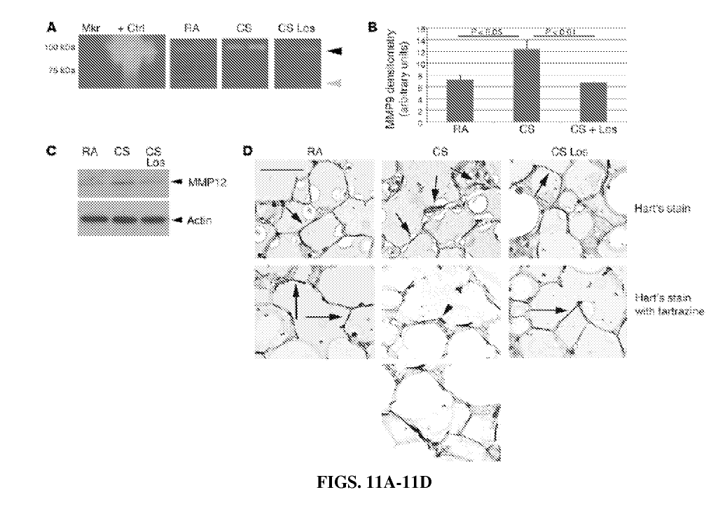

To further assess mechanisms by which elevated TGF-β might directly induce airspace enlargement, metalloprotease activation and matrix turnover was evaluated. Zymography showed increased MMP9, but not MMP2, activation with CS exposure compared with that after exposure to RAA MMP9 activation was normalized by losartan treatment (see, e.g., FIGS. 11A and 11B). Interestingly, a modest induction of MMP12 expression in the lungs of CS-exposed mice that was normalized by losartan treatment was also observed (see, e.g., FIG. 11C). Elastin fragmentation in the airspaces of CS-exposed mice was examined and discontinuous elastin staining with areas of clumping were found. This fragmentation was improved by losartan treatment (see, e.g., FIG. 11D). These data indicate that anti-TGF-β therapy may confer a protective milieu for the extracellular matrix in the CS-exposed lung. Without being bound by any particular theory, it is believed that both metalloprotease activation and apoptotic cell death are the likely underlying mechanisms for the CS-induced airspace enlargement, and according to the techniques herein, both are ameliorated by anti-TGF-β pharmacologic maneuvers.

### Example 6

### CS Alters Angiotensin Receptor Localization and Expression in the Murine Lung

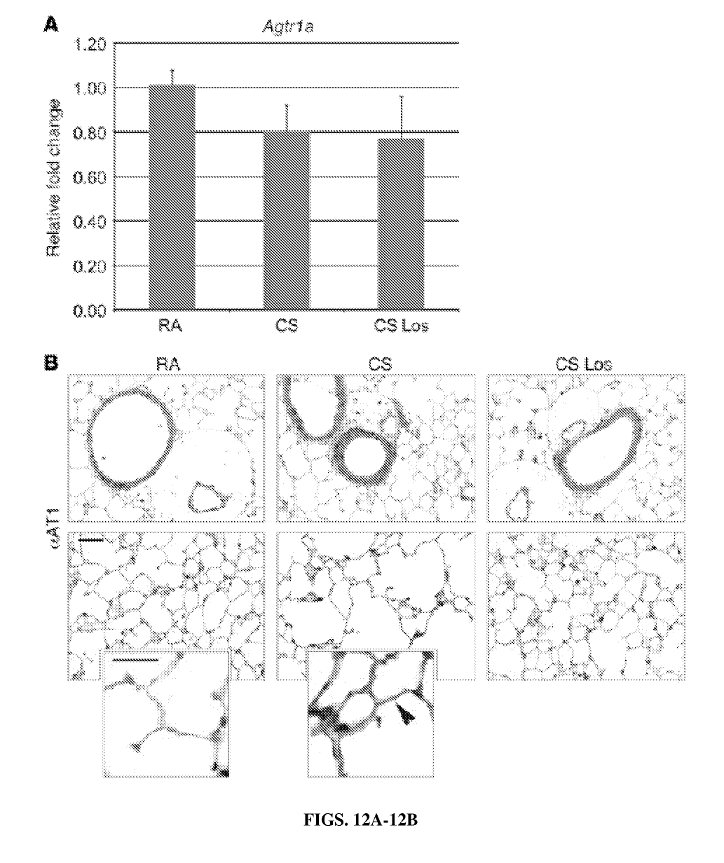

Because losartan is a specific angiotensin receptor type 1 (AT1) antagonist, it is possible that CS exposure dysregulated AT1 expression in a manner that enhanced the therapeutic utility of angiotensin receptor blockade. To assess this, AT1 receptor expression was examined using real-time PCR, which revealed that no differences in AT1 receptor expression were conferred by CS exposure (see, e.g., FIG. 12A). Angiotensin receptors are known to be expressed on lung epithelial cells, with AT1 localized primarily to the lung parenchyma (16, 17). Since receptor localization is an important factor in defining the mechanism of losartan's effects, immunohistochemistry for the AT1 receptor was performed on murine lungs subjected to RA, CS, and CS plus losartan. AT1 receptor was found to be localized to the alveolar wall and airway subepithelial mesenchymal layer (see, e.g., FIG. 12B). CS increased AT1 staining in the airspace walls, and this increase was normalized with losartan treatment (see, e.g., FIGS. 12B, and inset of 12B). Without being bound by any particular theory, it is believed that the therapeutic losartan effects observed in CS-exposed mice may partially reflect increased expression of angiotensin receptor 1 in the lung parenchyma that is induced by CS but normalized by losartan.

### Example 7

### Transcriptomic Signature of Therapeutic Effect with Losartan in CS Lung

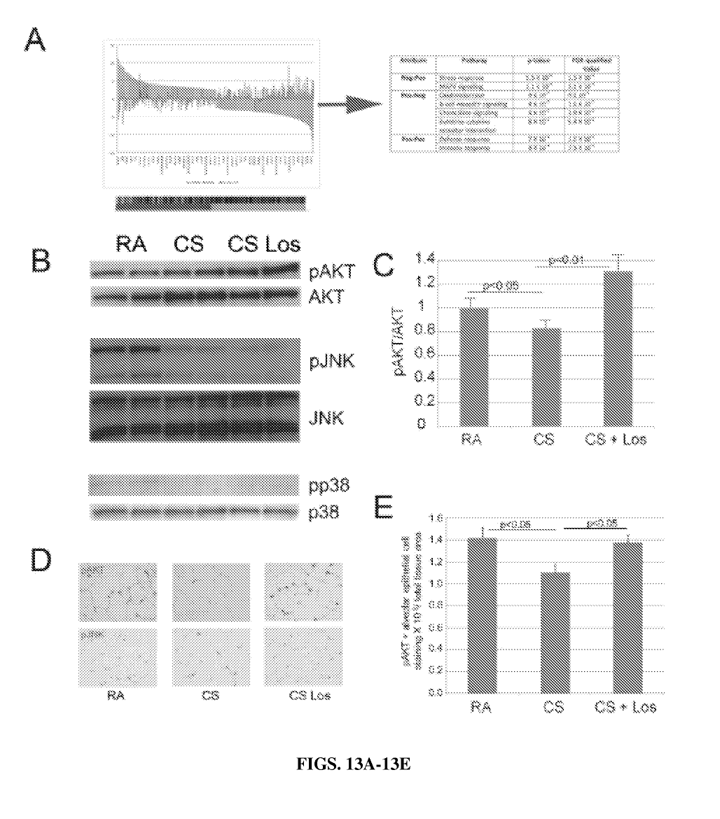

There is a dearth of rational therapies for COPD/emphysema in the conventional art. To identify non-intuitive pathways that could be exploited for therapeutic targeting, an expression profile analysis of lungs from mice exposed to RA, 2 months CS, or 2 months CS plus losartan was performed. A panel of genes dysregulated with CS and either further dysregulated or normalized when treated with losartan was generated (see, e.g., FIG. 13A). According to the techniques herein, genes induced or repressed with CS and then partially or fully normalized with losartan may represent pathways that contribute to the CS-induced injury phenotype. By contrast, genes primarily dysregulated with CS and then further dysregulated with losartan likely may reflect reparative pathways triggered with CS exposure and further reinforced by angiotensin receptor blockade. Interestingly, the stress response and MAPK pathway genes were downregulated with CS but induced with losartan treatment. Conversely, oxidoreductase, B cell receptor signaling, chemokine signaling, and cytokine receptor interaction pathways were induced with CS but repressed with losartan treatment. These data herein suggest that whereas survival pathways may be blunted with CS exposure but restored with losartan treatment, oxidative stress signaling and immune cell activation pathways are induced with CS and ameliorated with losartan treatment. Both expression profiles are consistent with the results described herein that losartan reduces CS-induced oxidative stress and inflammation (see, e.g., FIG. 9).

To further examine cell survival mechanisms that might be altered by CS but restored by losartan, the TGF-β-induced pathways that converge onto canonical survival kinase cascades (p21, p38, JNK, and PI3K/Akt) (18-21) were examined. In particular, the p21 (proapoptotic/antiapoptotic), p38 (proapoptotic), JNK (proapoptotic), and akt (antiapoptotic) pathways were assessed because they can be modulated by TGF-β. Since signaling measurements using total lung lysates are reflective of the composite of the multiple compartments present in the lung parenchyma, rather than the site of relevant activity, both immunoblotting and in situ surveys were used to assess prosurvival signaling with CS exposure and losartan treatment. No evidence of p21 induction or activation, respectively, was seen in CS-exposed lungs. Attenuated Akt, JNK, and p38 activation was observed by immunoblotting in CS-exposed lungs (see, e.g., FIG. 13B). However, only Akt activation was normalized by losartan treatment (see, e.g., FIG. 13C). These data suggest that losartan may improve airspace dimension by enhancing Akt-mediated prosurvival signaling and reducing alveolar apoptosis. To assess this, the distribution of akt staining in the lung was examined and found to be localized in the airspace epithelial cells (see, e.g., FIGS. 13D and 13E). The reduction in staining in the airspace compartment with CS indicated that the immunoblotting pattern reflected events at the site of known CS-induced lung pathology.

The role of TGF-β dysregulation in CS-induced COPD/emphysema is a controversial issue, given abundant but conflicting data showing evidence of both enhanced and reduced activity in the COPD lung. The data herein shows increased TGF-β activity in the airspaces of chronic CS-exposed mice and patients with mild COPD. Additionally, the techniques herein establish that pharmacologic inhibition of TGF-β signaling protects the murine lung from altered lung histology, impaired lung function, and a panel of injury measures that accompany CS-induced lung disease. Whereas emphysema was originally thought to solely require elastin destruction, the current pathogenetic schema incorporates additional mechanisms, such as cell death and oxidative stress injury (22, 23). Importantly, the pleiotropic effects of TGF-β signaling impact all of these contributing mechanisms. The techniques herein provide compelling preclinical evidence for the utility of TGF-β targeting for common and complex CS-promoted lung pathologies, such as COPD/emphysema and respiratory bronchiolitis.

TGF-β signaling incorporates a large family of ligands, cell surface receptors, and coreceptors that engage a complex but canonical cascade of intracellular mediators to modulate tissue morphogenesis and repair. TGF-β has multiple functions in the airspace, which is a compartment composed of multiple cell types of endodermal, mesenchymal, vascular, and hematopoietic lineage. The response to TGF-β in each of these cell types is distinct and context dependent (reviewed in ref. 24). The homeostatic level of TGF-β is well maintained, and the techniques herein indicate that interventions directed toward correcting excess TGF-β expression in either direction (e.g., high or low) are reasonable strategies. Although TGF-β can induce fibroblast cell differentiation into highly synthetic myofibroblasts and arguably transdifferentiation of epithelial cells into fibroblasts, the pathway can have prominent antiproliferative and proapoptotic effects in the epithelial compartment (14, 25). The results herein observed a prominent proapoptotic effect in the airspace epithelial compartment of CS-exposed lungs accompanying peribronchial fibrosis, which is consistent with a TGF-β-mediated profile. However, TGF-β effects in most tissues are dictated by both cellular context and signaling intensity, with a physiologic window defined by the optimal level of ambient ligand abundance and cellular capacity for response. According to the techniques herein, the selective epithelial and peribronchiolar response to TGF-β signaling suggests that chronic CS induces an elevation of TGF-β sufficient to compromise epithelial cell survival and promote submucosal fibrosis in the distal airway, but not to induce an interstitial fibrotic program. Of note, most TGF-β transgenic overexpression maneuvers in the lung result in exuberant pathway activation and therefore culminate in parenchymal fibrosis (26, 27). However, selective TGF-β-overexpressing mice, as well as nonfibrotic rodent injury models associated with elevated TGF-β levels, consistently show early airspace enlargement with variable components of mild fibrosis (28-30). Thus, the compartmentalized fibrotic effects of CS-induced TGF-β activity are fully consistent with other rodent models systems punctuated by injury-associated airspace enlargement.

Genetic data from multiple laboratories implicate disturbances in TGF-β signaling in COPD pathogenesis; however, the nature of the disturbance, too high or too low, is a subject of controversy. In several studies, TGFβ1 polymorphisms associate not only with the diagnosis of COPD but also with disease severity (31-34). However, other studies have not validated such associations (33, 35). Recently, polymorphisms in a TGF-β binding protein (LTBP) and a TGF-β coreceptor (betaglycan) were found to associate with distinct COPD-related subphenotypes (31, 36). Although a connection between TGF-β polymorphisms and serum levels was initially presumed based on a few publications, subsequent studies in larger and more heterogeneous populations have not consistently shown this association (37-40). Immunohistochemical studies of COPD lung specimens show evidence of enhanced TGF-β signaling predominantly in the airway compartment (41-43). Gene expression studies from lung specimens of patients with COPD demonstrate enhanced activation of TGF-β pathways that may well be stage and compartment dependent (44-46). Interestingly, selective animal models with defects in TGF-β signaling have also shown developmental or late-onset airspace enlargement (47-49). These seemingly conflicting findings suggest that a critical level of TGF-β signaling is required for airspace formation and maintenance and that disorders resulting in either marked excess or profound deficiency in TGF-β signaling translate into abnormal airspace architecture. Furthermore, the activation of compensatory mechanisms that serve to enhance TGF-β signaling might be operative in these models (50). Thus, dysregulated TGF-β signaling provides a unifying explanation for the divergent manifestations of COPD with cellular proliferation with fibrosis in terminal airways and apoptotic cell death in the alveolar compartment. The data herein provide, for the first time, evidence that enhanced TGF-β activity is not merely a signature of COPD, but that it contributes to disease pathogenesis.

The data herein demonstrate an intriguing and previously unreported airway epithelial phenotype that approximates the epithelial hyperplasia that can accompany a variety of airway insults, including CS (reviewed in ref. 51). Airway wall thickening is a complex pathology in clinical COPD, but seems to be a consequence of excessive TGF-β activation (42, 52). Whether submucosal matrix deposition, airway epithelial thickening, or mucus hypersecretion is the critical pathologic lesion that accounts for clinical obstruction is unknown (13). Murine models typically display modest airway wall remodeling in response to chronic CS, an observation that is thought to be a consequence of the anatomic and cell compositional differences between the rodent and the primate airway (53). Nonetheless, clinical hyperexpansion with air trapping is a direct consequence of the airway lesion and is associated with accelerated lung function decline in patients with emphysema (54). The data presented herein indicates that the increased lung volumes likely follow from the airway mucosal thickening. A recent small, short term, clinical trial of angiotensin receptor blockade in patients with COPD having pulmonary hypertension similarly showed improvement in lung hyperexpansion with 4 months of treatment (55). Thus, the data generated in our preclinical model approximate effects observed in small studies of this agent in a comparable clinical population.

The techniques herein provide that enhanced TGF-β is a therapeutic point of convergence for the inflammation, oxidative stress, cell death, and, importantly, that metalloprotease activation associated with chronic CS exposure. Metalloprotease activation causing matrix turnover is an important mechanism of COPD development and maintenance. Polymorphisms in MMP12 associate with reduced lung function in patients with COPD and children with asthma (31). Mice deficient in MMP12 are protected against CS-induced emphysema (56). However, the role of TGF-β signaling in metalloprotease expression and activation is highly contextual, with evidence of inductive effects on MMP9 and inhibitory effects on MMP12 (57-59). Further, reduced TGF-β signaling seems to punctuate some models of aging-related airspace enlargement, possibly secondary to both a temporally defined impairment in maintenance elastogenesis and elevated MMP12 expression (47, 74).

CS appears distinct from the above-described processes. Since TGF-β can induce MMP9 expression, and MMP9 can activate TGF-β, the pattern of MMP9 activation observed herein is consistent with a TGF-β-mediated process (60-62). In the aging-associated airspace enlargement models, TGF-β is thought to inhibit MMP12 expression in macrophages, which seems to contradict the results described herein; however, the seemingly paradoxical results herein may reflect a direct effect of CS exposure on the proposed regulatory scheme and/or the enhanced macrophage abundance in the lungs of CS-exposed mice (47). Even though airspace maintenance in the setting of CS exposure may converge upon known cell injury and cell death processes, the role of CS on prosurvival signaling in the airspace has not been well dissected. The data herein provide some insight into these cascades. Using a combination of whole tissue and in situ analysis, the techniques herein provide that reduced Akt signaling may be involved in the alveolar septal cell survival disturbance that culminates in enhanced cell death observed in the chronic CS model. It has been shown that Akt signaling is a critical mediator of airspace homeostasis in the setting of neonatal and adult hyperoxic injury (63, 64). Furthermore, several in vitro studies demonstrate that TGF-β directly inhibits Akt-mediated lung epithelial cell survival (65, 66). Without wishing to be bound by theory, a similar mechanism may be operative with chronic CS-induced lung injury.

As described in detail above, the techniques herein use a CS-induced emphysema model based on the AKR/J strain, rather than the C57BL/6 model used in the conventional art.

The techniques herein provide a murine model of CS-induced lung disease that manifests both airway wall thickening and airspace simplification after 2 months of smoke exposure. This model displays increased TGF-β signaling and oxidative stress and inflammation in the airway and alveolar compartments. Altered cell survival signaling culminates in increased alveolar cell death. More importantly, the systemic antagonism of TGF-β signaling with angiotensin receptor blockade (e.g., with losartan) was shown to normalize histology and reduce oxidative stress, cell death, and inflammation. Pulmonary function studies show improved lung mechanics with losartan treatment. An exploratory transcriptional survey implicates the involvement of immunomodulatory and stress response pathways in the therapeutic effects of losartan.

The results described herein above were obtained using the following methods and materials.

Mice:

Adult AKR/J mice were obtained from The Jackson Laboratory. These mice were housed in a facility accredited by the American Association of Laboratory Animal Care, and the animal studies were reviewed and approved by the institutional animal care and use committee of Johns Hopkins School of Medicine.

CS Exposure:

Six- to eight-week-old AKR/J male mice were divided into 3 groups. The control group was kept in a filtered air environment, and the experimental groups were subjected to CS or CS plus losartan in drinking water. CS exposure was carried out (2 hours per day, 5 days per week) by burning 2R4F reference cigarettes (University of Kentucky, Louisville, Ky., USA) using a smoking machine (Model TE-10; Teague Enterprises) for 6 to 7 weeks. The average concentration of total suspended particulates and carbon monoxide was 90 mg/m and 350 ppm, respectively, which was monitored on a routine basis.

Human Studies:

All human lung tissue from persons with COPD and atrisk controls were obtained, as anonymized samples, from the Lung Tissue Research Consortium (LTRC; http://www.nhlbi.nih.gov/resources/ltrc.htm), sponsored by the National, Heart Lung and Blood Institute. Based on spirometry and smoking history, the patients were designated as at-risk (>10 pack year history of smoking; normal spirometry) or as having moderate or severe COPD using Global Initiative for Chronic Obstructive Lung Disease (GOLD) criteria (moderate, GOLD, 2; forced expiratory volume at 1 second (FEV1), 50%-80% predicted; severe, GOLD, 3 and 4; FEV1, <50% predicted) (68). All smokers were former smokers.

Cell Treatment:

MLE12 cells (ATCC) were treated with CSE for 72 hours after serum starvation overnight. CSE was generated per standard protocol by the D'Amico laboratory, Johns Hopkins School of Medicine (69). Cell lysates were harvested and subjected to immunoblotting for psmad2 (Cell Signaling Technology).

Treatment Regimen:

The AT1 selective antagonist losartan (Merck Co.) was diluted into drinking water at concentrations of 3 mg/kg (low dose) and 30 mg/kg (high dose). Panselective TGF-β-neutralizing antibody (R&D Systems) was administered by intraperitoneal injection according to published protocol (70). Isotype-matched control antibody (R&D Systems) was administered to control mice as described above.

Morphology and Histology:

Three to five mice of each genotype were studied at the noted ages. For histologic and morphometric analyses, mouse lungs were inflated at a pressure of 25 cm H2O and fixed with 4% PFA in low molecular weight agarose. The lungs were equilibrated in cold 4% PFA overnight, sectioned, and then embedded in paraffin wax. Sections were cut at 5 μm and either stained with H&E or processed for immunohistochemistry. For the human lung samples, 2-3 slides from each patient or control were used for analysis.

Morphometry and Histochemistry:

Mean linear intercept measurements were performed on H&E-stained sections taken at intervals throughout both lungs. Slides were coded, captured by an observer, and masked for identity for the groups. Ten to fifteen images per slide were acquired at ×20 magnification and transferred to a computer screen. Mean chord lengths and mean linear intercepts were assessed by automated morphometry with a macro-operation performed by Metamorph Imaging Software (Universal Imaging, Molecular Devices). Mean airway thickness was measured directly using microscope-captured images at ×40 magnification. Hart's staining was performed per published protocol using either van Gieson or tartrazine counterstaining (71).

Immunoblotting:

Whole lung lysates were extracted in M-Per buffer from Pierce. Protein concentrations were determined using the Bio-Rad Protein Assay. Aliquots of 30-50 μg protein were boiled and then loaded onto Tris-HCL gels and transferred electrophoretically to nitrocellulose membranes. Membranes were incubated with the primary antibody for 1 hour at room temperature. Detection was performed by the Pierce West Dura ECL Detection System. Primary antibodies and dilutions were as follows: β-actin (rabbit polyclonal, 1:1,000; Abcam), p38 (rabbit polyclonal, 1:1,000; Cell Signaling Technology), pp 38 (goat polyclonal, 1:200; Cell Signaling Technology), ERK1 (rabbit polyclonal, 1:1,000; Cell Signaling Technology), pERK1 (rabbit polyclonal, 1:1,000; Cell Signaling Technology), JNK (rabbit polyclonal, 1:1,000; Cell Signaling Technology), and pJNK (rabbit polyclonal, 1:1,000; Cell Signaling Technology).

Immunohistochemistry:

Tissue sections were deparaffinized and rehydrated in an ethanol series. Sections were blocked for non-specific binding with 3% normal serum from chicken and incubated with the primary antibodies for 1 hour at room temperature. For immunofluorescence, sections were then incubated with secondary antibodies at 1:200 for 30 minutes at room temperature (Molecular Probes). Sections were counterstained with 4′,6′-diamidinio-2-phenylindole (DAPI) and mounted with Vectashield hard set mounting medium (Vector Labs). Briefly, after incubation with the primary antibody overnight at 4° C., slides were washed with PBST, incubated with an appropriate biotinylated secondary antibody (Jackson ImmunoResearch Inc.), and developed by using ABC and DAB detection reagents (Vector Laboratories). Antibodies were used at the following concentrations: Ki67 (1:50; Santa Cruz Biotechnology Inc.), nitrotyrosine (Abcam), Mac3 (BD Biosciences), CD45R (Santa Cruz Biotechnology Inc.), psmad2 (Cell Signaling Technology), TUNEL (1:25; Abcam), JNK/pJNK (Cell Signaling Technology), Akt/pAkt (Cell Signaling Technology), LAP— TGF-β1 (R&D Systems), CTGF (Abcam), Angiotensin type 1 receptor (Santa Cruz Biotechnology Inc.), and active caspase-3 (Abcam).

Measurement of Mouse Lung Mechanics:

Mice were anesthetized with a ketamine (90 mg/kg)/xylazine (18 mg/kg) mixture. Once sedated, a tracheostomy was performed, and a cannula (18G) was inserted and connected to a constant flow ventilator as previously described (72). Quasistatic PV curves were performed as previously reported (73). Details regarding protocol are in the Supplemental Methods.

Statistics:

One-way ANOVA with Tukey's post-hoc test or Kruskal-Wallis nonparametric analysis with a Dunnett's post-hoc test were used to determine differences among groups. When 2 groups were compared, an unpaired, 2-tailed Student's t-test or a Wilcoxon rank-sum test was used. Values for all measurements were expressed as mean±SEM, and P values for significance were less than 0.05. The number of samples or animals in each group is indicated in the figure legends or text.

Study Approval:

For the LTRC specimens, all patients provided informed consent to the LTRC. The IRB-exempt status for these studies was confirmed with the Johns Hopkins Office of Human Subjects Research (study no. NA_{—}0051734).

ELISA Analysis:

The active mature fragment of TGFβ was measured using the R&D Duoset assay (Cat#DY1679). Polystyrene plates (Maxisorb; Nunc) were coated with capture antibody in PBS overnight at 25° C. The plates were washed 4 times with 50 mM Tris, 0.2% Tween-20, pH 7.0-7.5 and then blocked for 90 minutes at 25° C. with assay buffer (PBS containing 4% BSA (Sigma) and 0.01% Thimerosal, pH 7.2-7.4). The plates were washed 4 times and 500 assay buffer was added to each along with 500 of sample or standard prepared in assay buffer and incubated at 37° C. for 2 h. The plates were washed 4 times and 1000 of biotinylated detecting antibody in assay buffer was added and incubated for 1 h at 25° C. After washing the plate 4 times strepavidin-peroxidase polymer in casein buffer (RDI) was added and incubated at 25° C. for 30 min. The plate was washed 4 times and 1000 of commercially prepared substrate (TMB; Neogen) was added and incubated at 25° C. for approximately 10-30 min. The reaction was stopped with 100 μl 2N HCl and the A450 (minus A650) was read on a microplate reader (Molecular Dynamics). A curve was fit to the standards using a computer program (SoftPro; Molecular Dynamics) and cytokine concentration in each sample was calculated from the standard curve equation. Levels below the assay range should be interpreted as “Low” (below the lower detection limit). Because of the shape of the standard curve, negative values are occasionally calculated for some samples. These should also be interpreted as “undetectable.” Values above the range are calculated by extrapolation and thus may not be accurate. Those samples that are above or below the range were marked in the “Inrange” column of the results as “High.”

Zymography:

Lung tissue lysates were prepared in a cold room at 4 C. Tissue was homogenized in 50 μL PBS and centrifuged at 14000 RPM for 20 min. The supernatant was removed and used as sample lysates. Fifty μg of lung lysates were loaded on a 10% Criterion Zymography Precast Gel (Biorad) and run at 120V. Twenty-five μL of recombinant mouse MMP9 protein (R&D Systems, Minneapolis, Minn.) was loaded as a positive control. The gel was soaked in 1× Renaturing Buffer (Biorad) twice for 30 minutes each at room temperature and incubated in 1× Development Buffer (Biorad) overnight at 37C. The gels were stained with Coomassie Brilliant Blue R-250 Staining Solution (Biorad), followed by 1× Destain Coomassie R-250 Solution (Biorad) until a clear band appeared against a blue background.

Measurement of Mouse Lung Mechanics.

After being connected they were paralyzed with Succinylcholine (75 mg/kg) and ventilated with a tidal volume of 0.2 mL of 100% oxygen at a rate of 150 breathes/min, with a positive end expiratory pressure (PEEP) of 3 cm H2O. A deep inspiration (to 30 cmH2O for 5 sec) was given and then the animal was returned to normal ventilation. One minute later Rrs and Ers were measured (74). After determination of Rrs and Ers, ventilation was stopped, and the tracheal cannula was occluded for 4 min, which led to complete degassing of the lungs by absorption atelectasis. Quasi-static PV curves were performed as previously reported (75). Quasistatic compliance of the respiratory system was computed from the P-V relationships as the slope of the deflation limb between 3 and 8H2O, which is where the curves are most linear. Real-Time PCR: Total RNA isolated from lung tissues was treated with DNase and reverse-transcribed using a first-strand DNA sysnthesis kit from Invitrogen. The PCR was performed on an ABI Fast 7500 System (Applied Biosystems, Foster City, Calif.). TaqMan probes for the respective genes were custom-generated by Applied Biosystems based on the sequences in the IIlumina array and used per manufacturer's instructions. The expression levels of target genes were determined in triplicate from the standard curve and normalized to Gapdh mRNA level.

RNA Extraction and Illumina Chip Hybridization:

Total RNA was extracted from the designated murine lungs, six in each treatment group, using the Trizol Reagent method (Invitrogen, Carlsbad, Calif. 92008, cat. no. 15596-026). Additional purification was performed on RNAeasy columns (Qiagen, Valencia, Calif. 913555, cat. no. 74104). The quality of total RNA samples was assessed using an Agilent 2100 Bioanalyzer (Agilent Technologies, Palo Alto, Calif.). The six RNA samples from each time point were pooled into two groups comprised of three murine specimens. RNA samples were labeled according to the chip manufacturers recommended protocols. In brief, for Illumina, 0.5 μg of total RNA from each sample was labeled by using the Illumina TotalPrep RNA Amplification Kit (Ambion, Austin, Tex. 78744-1832, cat. no. IL1791) in a process of cDNA synthesis and in vitro transcription. Single stranded RNA (cRNA) was generated and labeled by incorporating biotin-16-UTP (Roche Diagnosics GmbH, Mannheim, Germany, cat. no. 11388908910). 0.85 ugs of biotin-labeled cRNA was hybridized (16 hours) to Illumina's Sentrix MouseRef-8 Expression BeadChips (Illumina, San Diego, Calif. 92121-1975, cat. no. BD-26-201). The hybridized biotinylated cRNA was detected with streptavidin-Cy3 and quantitated using Illumina's BeadStation 500GX Genetic Analysis Systems scanner. The complete data set has been submitted and is currently available in the Gene Expression Omnibus database (http://www.ncbi.nlm.nih.gov/geo/; accession number: GSE33561).

Microarray Analysis:

DAVID Analysis (NIAID) was used to analyze expression profile pathway data from the various treatment groups (76). DAVID provides typical batch annotation and gene-GO term enrichment analysis to highlight the most relevant GO terms associated with a given gene list. Extended annotation includes GO terms, protein-protein interactions, protein functional domains, disease associations, bio-pathways, sequence general features, homologies, gene functional summaries, gene tissue expressions, literatures, etc. In the DAVID annotation system, the Fisher Exact test is adopted to measure the gene-enrichment in annotation terms and generate significance estimates (p-values).

### REFERENCES

1. Barnes P J. New treatments for COPD. Nat Rev Drug Discov. 2002; 1(6):437-446.
2. Habashi J P, et al. Losartan, an AT1 antagonist, prevents aortic aneurysm in a mouse model of Marfan syndrome. Science. 2006; 312(5770):117-121.
3. Neptune E R, et al. Dysregulation of TGF-beta activation contributes to pathogenesis in Marfan syndrome. Nat. Genet. 2003; 33(3):407-411.
4. Lee C G, Kang H R, Homer R J, Chupp G, Elias J A. Transgenic modeling of transforming growth factor-beta(1): role of apoptosis in fibrosis and alveolar remodeling. Proc Am Thorac Soc. 2006; 3(5):418-423.
5. Sheppard D. Transforming growth factor beta: a central modulator of pulmonary and airway inflammation and fibrosis. Proc Am Thorac Soc. 2006; 3(5):413-417.
6. Konigshoff M, Kneidinger N, Eickelberg O. TGFbeta signaling in COPD: deciphering genetic and cellular susceptibilities for future therapeutic regimen. Swiss Med. Wkly. 2009; 139(39-40):554-563.
7. Kagami S, Border W A, Miller D E, Noble N A. Angiotensin II stimulates extracellular matrix protein synthesis through induction of transforming growth factor-beta expression in rat glomerular mesangial cells. J Clin Invest. 1994; 93(6):2431-2437.
8. Zhou Y, Poczatek M H, Berecek K H, Murphy-Ullrich J E. Thrombospondin 1 mediates angiotensin II induction of TGF-beta activation by cardiac and renal cells under both high and low glucose conditions. Biochem Biophys Res Commun. 2006; 339(2):633-641.
9. Li X, Rayford H, Uhal B D. Essential roles for angiotensin receptor AT1a in bleomycin-induced apoptosis and lung fibrosis in mice. Am J. Pathol. 2003; 163(6):2523-2530.
10. Marshall R P, et al. Angiotensin II and the fibroproliferative response to acute lung injury. Am J Physiol Lung Cell Mol Physiol. 2004; 286(1):L156-L164.
11. Guerassimov A, et al. The development of emphysema in cigarette smoke-exposed mice is strain dependent. Am J Respir Crit Care Med. 2004; 170(9):974-980.
12. Calvi C L, et al. Critical transition in tissue homeostasis accompanies murine lung senescence. PLoS One. 2011; 6(6):e20712.
13. Hogg J C, et al. The nature of small-airway obstruction in chronic obstructive pulmonary disease. N Engl J Med. 2004; 350(26):2645-2653.
14. Marwick J A, Kirkham P, Gilmour P S, Donaldson K, Mac N W, Rahman I. Cigarette smoke-induced oxidative stress and TGF-beta1 increase p21waf1/cip1 expression in alveolar epithelial cells. Ann NY Acad Sci. 2002; 973:278-283.
15. Stockley R A, Mannino D, Barnes P J. Burden and pathogenesis of chronic obstructive pulmonary disease. Proc Am Thorac Soc. 2009; 6(6):524-526.
16. Pavel J, et al. Increased angiotensin II AT1 receptor mRNA and binding in spleen and lung of AT2 receptor gene disrupted mice. Regul Pept. 2009; 158(1-3):156-166.
17. Bullock G R, et al. Distribution of type-1 and type-2 angiotensin receptors in the normal human lung and in lungs from patients with chronic obstructive pulmonary disease. Histochem Cell Biol. 2001; 115(2): 117-124.
18. Conery A R, Cao Y, Thompson E A, Townsend C M Jr, Ko T C, Luo K. Akt interacts directly with Smad3 to regulate the sensitivity to TGF-beta induced apoptosis. Nat Cell Biol. 2004; 6(4):366-372.
19. Undevia N S, Dorscheid D R, Marroquin B A, Gugliotta W L, Tse R, White S R. Smad and p38-MAPK signaling mediates apoptotic effects of transforming growth factor-beta1 in human airway epithelial cells. Am J Physiol Lung Cell Mol Physiol. 2004; 287(3):L515-L524.
20. Kang H R, Cho S J, Lee C G, Homer R J, Elias J A. Transforming growth factor (TGF)-beta1 stimulates pulmonary fibrosis and inflammation via a Bax-dependent, bid-activated pathway that involves matrix metalloproteinase-12. J Biol Chem. 2007; 282(10):7723-7732.
21. Kaur S, Wang F, Venkatraman M, Arsura M. Xlinked inhibitor of apoptosis (XIAP) inhibits c-Jun N-terminal kinase 1 (JNK1) activation by transforming growth factor beta1 (TGF-beta1) through ubiquitin-mediated proteosomal degradation of the TGF-beta1-activated kinase 1 (TAK1). J Biol Chem. 2005; 280(46):38599-38608.
22. Chung K F, Adcock I M. Multifaceted mechanisms in COPD: inflammation, immunity, and tissue repair and destruction. Eur Respir J. 2008; 31(6):1334-1356.
23. Sharafkhaneh A, Hanania N A, Kim V. Pathogenesis of emphysema: from the bench to the bedside. Proc Am Thorac Soc. 2008; 5(4):475-477.
24. Morty R E, Konigshoff M, Eickelberg 0. Transforming growth factor-beta signaling across ages: from distorted lung development to chronic obstructive pulmonary disease. Proc Am Thorac Soc. 2009; 6(7):607-613.
25. Yamasaki M, et al. P21 regulates TGF-beta1-induced pulmonary responses via a TNF-alpha signaling pathway. Am J Respir Cell Mol Biol. 2008; 38(3):346-353.
26. Lee C G, et al. Early growth response gene 1-mediated apoptosis is essential for transforming growth factor beta1-induced pulmonary fibrosis. J Exp Med. 2004; 200(3):377-389.
27. Sime P J, Sarnstrand B, Xing Z, Graham F, Fisher L, Gauldie J. Adenovirus-mediated gene transfer of the proteoglycan biglycan induces fibroblastic responses in the lung. Chest. 1997; 111(6 suppl):1375.
28. Zhou L, Dey C R, Wert S E, Whitsett J A. Arrested lung morphogenesis in transgenic mice bearing an SP-C-TGF-beta 1 chimeric gene. Dev Biol. 1996; 175(2):227-238.
29. Lee C G, Cho S, Homer R J, Elias J A. Genetic control of transforming growth factor-beta1-induced emphysema and fibrosis in the murine lung. Proc Am Thorac Soc. 2006; 3(6):476-477.
30. Nakanishi H, Sugiura T, Streisand J B, Lonning S M, Roberts J D Jr. TGF-beta-neutralizing antibodies improve pulmonary alveologenesis and vasculogenesis in the injured newborn lung. Am J Physiol Lung Cell Mol Physiol. 2007; 293(1):L151-L161.
31. Hunninghake G M, et al. MMP12, lung function, and COPD in high-risk populations. N Engl J Med. 2009; 361(27):2599-2608.
32. Cho M H, et al. Cluster analysis in severe emphysema subjects using phenotype and genotype data: an exploratory investigation. Respir Res. 2010; 11:30.
33. van Diemen C C, Postma D S, Vonk J M, Bruinenberg M, Nolte I M, Boezen H M. Decorin and TGF-beta1 polymorphisms and development of COPD in a general population. Respir Res. 2006; 7:89.
34. Su Z G, Wen F Q, Feng Y L, Xiao M, Wu X L. Transforming growth factor-beta1 gene polymorphisms associated with chronic obstructive pulmonary disease in Chinese population. Acta Pharmacol Sin. 2005; 26(6):714-720.
35. Yoon H I, et al. Lack of association between COPD and transforming growth factor-beta1 (TGFB1) genetic polymorphisms in Koreans. Int J Tuberc Lung Dis. 2006; 10(5):504-509.
36. Hersh C P, et al. Transforming growth factor-beta receptor-3 is associated with pulmonary emphysema. Am J Respir Cell Mol. Biol. 2009; 41(3):324-331.
37. Carrieri G, et al. The G/C915 polymorphism of transforming growth factor beta1 is associated with human longevity: a study in Italian centenarians. Aging Cell. 2004; 3(6):443-448.
38. Grainger D J, et al. Genetic control of the circulating concentration of transforming growth factor type beta1. Hum Mol Genet. 1999; 8(1):93-97.
39. Rodriguez-Rodriguez E, et al. Serum levels and genetic variation of TGF-beta1 are not associated with Alzheimer's disease. Acta Neurol Scand. 2007; 116(6):409-412.
40. Suthanthiran M, et al. Transforming growth factorbeta 1 hyperexpression in African-American hypertensives: A novel mediator of hypertension and/or target organ damage. Proc Natl Acad Sci USA. 2000; 97(7):3479-3484.
41. Beghe B, et al. Transforming growth factor-beta type II receptor in pulmonary arteries of patients with very severe COPD. Eur Respir J. 2006; 28(3):556-562.
42. Takizawa H, et al. Increased expression of transforming growth factor-beta1 in small airway epithelium from tobacco smokers and patients with chronic obstructive pulmonary disease (COPD). Am J Respir Crit Care Med. 2001; 163(6):1476-1483.
43. Aubert J D, Dalal B I, Bai T R, Roberts C R, Hayashi S, Hogg J C. Transforming growth factor beta 1 gene expression in human airways. Thorax. 1994; 49(3):225-232.
44. Ning W, et al. Comprehensive gene expression profiles reveal pathways related to the pathogenesis of chronic obstructive pulmonary disease. Proc Natl Acad Sci USA. 2004; 101(41):14895-14900.
45. Matsumoto M, et al. Exogenous heat shock protein-70 inhibits cigarette smoke-induced intimal thickening. Am J Physiol Regul Integr Comp Physiol. 2008; 295(4):R1320-R1327.
46. Gosselink J V, et al. Differential expression of tissue repair genes in the pathogenesis of chronic obstructive pulmonary disease. Am J Respir Crit Care Med. 2010; 181(12):1329-1335.
47. Morris D G, et al. Loss of integrin alpha(v) beta6-mediated TGF-beta activation causes Mmp12-dependent emphysema. Nature. 2003; 422(6928):169-173.
48. Bonniaud P, et al. Smad3 null mice develop airspace enlargement and are resistant to TGFbeta-mediated pulmonary fibrosis. J Immunol. 2004; 173(3):2099-2108.
49. Chen H, et al. Abnormal mouse lung alveolarization caused by Smad3 deficiency is a developmental antecedent of centrilobular emphysema. Am J Physiol Lung Cell Mol Physiol. 2005; 288(4):L683-L691.
50. Farkas L, et al. Cigarette smoke exposure aggravates airspace enlargement and alveolar cell apoptosis in Smad3 knockout mice. Am J Physiol Lung Cell Mol Physiol. 2011; 301(4):L391-L401.
51. Puchelle E, Zahm J M, Tournier J M, Coraux C. Airway epithelial repair, regeneration, and remodeling after injury in chronic obstructive pulmonary disease. Proc Am Thorac Soc. 2006; 3(8):726-733.
52. Churg A, Wang R D, Wright J L. Cigarette smoke causes small airway remodeling by direct growth factor induction and release. Proc Am Thorac Soc. 2006; 3(6):493.
53. Wright J L, Cosio M, Churg A. Animal models of chronic obstructive pulmonary disease. Am J Physiol Lung Cell Mol Physiol. 2008; 295(1):L1-L15.
54. Sturton G, Persson C, Barnes P J. Small airways: an important but neglected target in the treatment of obstructive airway diseases. Trends Pharmacol Sci. 2008; 29(7):340-345.
55. Andreas S, et al. Angiotensin II blockers in obstructive pulmonary disease: a randomised controlled trial. Eur Respir J. 2006; 27(5):972-979.
56. Hautamaki R D, Kobayashi D K, Senior R M, Shapiro S D. Requirement for macrophage elastase for cigarette smoke-induced emphysema in mice. Science. 1997; 277(5334):2002-2004.
57. Feinberg M W, et al. Transforming growth factor-beta 1 inhibits cytokine-mediated induction of human metalloelastase in macrophages. J Biol Chem. 2000; 275(33):25766-25773.
58. Behzadian M A, Wang X L, Windsor L J, Ghaly N, Caldwell R B. TGF-beta increases retinal endothelial cell permeability by increasing MMP-9: possible role of glial cells in endothelial barrier function. Invest Ophthalmol Vis Sci. 2001; 42(3):853-859.
59. Li D Q, Lokeshwar B L, Solomon A, Monroy D, Ji Z, Pflugfelder S C. Regulation of MMP-9 production by human corneal epithelial cells. Exp Eye Res. 2001; 73(4):449-459.
60. Safina A, Vandette E, Bakin A V. ALKS promotes tumor angiogenesis by upregulating matrix metalloproteinase-9 in tumor cells. Oncogene. 2007; 26(17):2407-2422.
61. Tian Y C, et al. Epidermal growth factor and transforming growth factor-beta1 enhance H K-2 cell migration through a synergistic increase of matrix metalloproteinase and sustained activation of ERK signaling pathway. Exp Cell Res. 2007; 313(11):2367-2377.
62. Yu Q, Stamenkovic I. Cell surface-localized matrix metalloproteinase-9 proteolytically activates TGFbeta and promotes tumor invasion and angiogenesis. Genes Dev. 2000; 14(2):163-176.
63. el-Remessy A B, Bartoli M, Platt D H, Fulton D, Caldwell R B. Oxidative stress inactivates VEGF survival signaling in retinal endothelial cells via PI 3-kinase tyrosine nitration. J Cell Sci. 2005; 118(pt 1):243-252.
64. Alphonse R S, et al. Activation of akt protects alveoli from neonatal oxygen-induced lung injury. Am J Respir Cell Mol Biol. 2011; 44(2):146-154.
65. Ju E M, et al. Apoptosis of mink lung epithelial cells by co-treatment of low-dose staurosporine and transforming growth factor-beta1 depends on the enhanced TGF-beta signaling and requires the decreased phosphorylation of PKB/Akt. Biochem Biophys Res Commun. 2005; 328(4):1170-1181.
66. Solovyan V T, Keski-Oja J. Proteolytic activation of latent TGF-beta precedes caspase-3 activation and enhances apoptotic death of lung epithelial cells. J Cell Physiol. 2006; 207(2):445-453.
67. Lee K M, Renne R A, Harbo S J, Clark M L, Johnson R E, Gideon K M. 3-week inhalation exposure to cigarette smoke and/or lipopolysaccharide in AKR/J mice. Inhal Toxicol. 2007; 19(1):23-35.
68. Pauwels R A, Buist A S, Calverley P M, Jenkins C R, Hurd S S. Global strategy for the diagnosis, management, and prevention of chronic obstructive pulmonary disease. NHLBI/WHO Global Initiative for Chronic Obstructive Lung Disease (GOLD) Workshop summary. Am J Respir Crit Care Med. 2001; 163(5):1256-1276.
69. Damico R, et al. p53 mediates cigarette smoke induced apoptosis of pulmonary endothelial cells: inhibitory effects of macrophage migration inhibitor factor. Am J Respir Cell Mol Biol. 2011; 44(3):323-332.
70. Cohn R D, et al. Angiotensin II type 1 receptor blockade attenuates TGF-beta-induced failure of muscle regeneration in multiple myopathic states. Nat Med. 2007; 13(2):204-210.
71. Pierce R A, Albertine K H, Starcher B C, Bohnsack J F, Carlton D P, Bland R D. Chronic lung injury in preterm lambs: disordered pulmonary elastin deposition. Am J Physiol. 1997; 272(3 Pt 1):L452-L460.
72. Ewart S, Levitt R, Mitzner W. Respiratory system mechanics in mice measured by end-inflation occlusion. J Appl Physiol. 1995; 79(2):560-566.
73. Soutiere S E, Mitzner W. On defining total lung capacity in the mouse. J Appl Physiol. 2004; 96(5):1658-1664.
74. Ewart, S., Levitt, R., and Mitzner, W. 1995. Respiratory system mechanics in mice measured by end-inflation occlusion. J Appl Physiol 79:560-566.
75. Soutiere, S. E., and Mitzner, W. 2004. On defining total lung capacity in the mouse. J Appl Physiol 96:1658-1664.
76. Huang da, W., Sherman, B. T., and Lempicki, R. A. 2009. Systematic and integrative analysis of large gene lists using DAVID bioinformatics resources. Nat Protoc 4:44-57.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for treating or preventing lung cell damage associated with cigarette smoke or other environmental exposure, the method comprising contacting a cell with an effective amount of an agent that inhibits TGF-β signaling.

2. The method of claim 1, wherein the cell is a pulmonary cell, endothelial cell, pulmonary endothelial cell, smooth muscle cell, ciliated and unciliated epithelial cell, and/or alveolar cell.

3. The method of claim 1, wherein the cell is contacted for a time sufficient to improve lung architecture or lung function.

4. The method of claim 3, wherein the time is at least about 3, 6, 9, 12, 18, 24 months or more.

5. The method of claim 1, wherein the agent is a small compound, polypeptide, polynucleotide, or inhibitory nucleic acid molecule.

6. A method of preventing or reducing cell death associated with cigarette smoke-induced cell injury or other environmental exposure, the method comprising contacting a cell at risk of cell death with an agent that inhibits TGF-β signaling, thereby preventing or reducing cell death relative to an untreated control cell.

7. The method of claim 6, wherein the cell death is necrotic or apoptotic.

8. A method of treating or preventing chronic obstructive pulmonary disease (COPD), emphysema, and other symptoms associated with lung tissue injury in a subject at risk thereof, the method comprising administering to the subject an effective amount of an agent that inhibits TGF-β signaling.

9. A method of treating or preventing a lung disease selected from the group consisting of acquired lung disease, lung conditions associated with cigarette smoke or other environmental exposures, and lung manifestations associated with matrix disorders, the method comprising administering to the subject an effective amount of an agent that inhibits TGF-β signaling and/or an angiotensin receptor type 1 blocker/inhibitor.

10. The method of claim 4, wherein the acquired lung disease is selected from the group consisting of chronic obstructive pulmonary disease (COPD), bronchopulmonary dysplasia (BPD), emphysema, asthma, and aging related lung dysfunction.

11. The method of claim 4, wherein the matrix disorder is selected from the group consisting of Ehlers Danlos Syndrome, Cutis Laxa, and fibrosis.

12. The method of claim 1, wherein the method prevents or ameliorates alveolar injury, airway epithelial hyperplasia, and lung fibrosis.

13. The method of claim 1, wherein the agent is a TGF-β antagonist selected from the group consisting TGF-β antibodies, small compounds that modulate TGF-β signaling, inhibitory nucleic acids targeting TGF-β, and Alk1 and/or Alk5 inhibitors or angiotensin receptor type 1 blockers/inhibitors selected from the group consisting of Losartan, Telmesartan, Irbesartan, Candesartan, Eprosartan, Olmesartan, and Valsartan.

14. The method of claim 1 wherein the method prevents cell death or cell damage of a pulmonary cell, endothelial cell, pulmonary endothelial cell, smooth muscle cell, ciliated and unciliated epithelial cell, and/or alveolar cell.

15. The method of claim 1, wherein the agent is administered before, during, or after cigarette smoke-induced cell injury.

16. The method of claim 1, wherein the agent is administered to subjects having or at risk for developing a lung disease selected from the group consisting of Ehlers Danlos Syndrome, Cutis Laxa, acquired lung disease, bronchopulmonary dysplasia (BPD), aging related lung dysfunction, chronic obstructive pulmonary disease (COPD), emphysema, asthma, alveolar injury, airway epithelial hyperplasia, or fibrosis.

17. The method of claim 1, wherein the agent is formulated for delivery by inhalation.

18. A composition formulated for inhalation, the composition comprising an effective amount of an agent that inhibits TGF-β selected from the group consisting TGF-β antibodies, small compounds that modulate TGF-β signaling, inhibitory nucleic acids targeting TGF-β, and Alk1 and/or Alk5 inhibitors in an excipient formulated for delivery to the lung.

19. A device for delivering an aerosol to the lung comprising the composition of claim 18.

20. A composition formulated for inhalation, the composition comprising an effective amount of an angiotensin receptor type 1 blockers/inhibitor selected from the group consisting of Losartan, Telmesartan, Irbesartan, Candesartan, Eprosartan, Olmesartan, and Valsartan in an excipient formulated for delivery to the lung.

21. A device for delivering an aerosol to the lung comprising the composition of claim 20.

22. A packaged pharmaceutical comprising a therapeutically effective amount of an agent that inhibits TGF-β selected from the group consisting TGF-β antibodies, small compounds that modulate TGF-β signaling, inhibitory nucleic acids targeting TGF-β, and Alk1 and/or Alk5 inhibitors and instructions for use.

23. A packaged pharmaceutical comprising a therapeutically effective amount of an agent that is an angiotensin receptor type 1 blockers or inhibitor selected from the group consisting of Losartan, Telmesartan, Irbesartan, Candesartan, Eprosartan. Olmesartan, and Valsartan labeled for use in preventing or treating cigarette smoke-induced cell injury.

24. A kit for the amelioration of treating or preventing cigarette smoke-induced cell injury comprising an agent that inhibits TGF-β signaling and written instructions for use of the kit.

25. A packaged pharmaceutical comprising a therapeutically effective amount of an agent that is an angiotensin receptor type 1 blocker or inhibitor selected from the group consisting of Losartan, Telmesartan, Irbesartan, Candesartan, Eprosartan, Olmesartan, and Valsartan labeled for use in preventing or treating cigarette smoke-induced cell injury.
